(19) United States
(12) Patent Application Publication
(10) Pub. No.: US 2010/0029646 A1
(43) Pub. Date: Feb. 4, 2010

(54) PRODRUGS OF DIPHENYL OX-INDOL-2-ONE COMPOUNDS

(75) Inventors: Mette Knak Christensen, Holte (DK); Fredrik Bjoerkling, Helsingborg (SE); Martins Ikaunieks, Riga (LV); Andrei Zaichenko, Riga (LV); Vija Gailite, Riga (LV); Einars Loza, Riga (LV); Ivars Kalvinsh, Riga (LV); Marina Madre, Riga (LV)

Correspondence Address: DARBY & DARBY P.C., P.O. BOX 770, Church Street Station, New York, NY, 10008-0770, US

(73) Assignee: Topo Target A/S, Copenhagen (DK)

(21) Appl. No.: 12/518,545

(22) PCT Filed: Dec. 11, 2007
(86) PCT No.: PCT/EP2007/010805

§ 371 Date: Oct. 5, 2009

Related U.S. Application Data
(60) Provisional application No. 60/869,428, filed on Dec. 11, 2006.

(51) Int. Cl. A61K 31/404; C07D 209/34; C07D 403/06; C07D 413/06; A61P 35/00; A61K 31/496; A61K 31/5377

(52) U.S. Cl. 514/235.2; 548/487; 544/144; 514/418; 544/373; 514/254.09

(57) ABSTRACT

The present application discloses prodrugs of substituted 3,3-diphenyl-1,3-dihydro-indol-2-one compounds having the general formula (I). The 4-position of the phenyl moieties and the N-position of the indole represent useful handles for the introduction of particular prodrug groups, in particular those comprising an amino acid moiety. The prodrug compounds are believed to be useful for the treatment of cancer in a mammal, possibly in combination with one or more other chemotherapeutic agents. The application also discloses the compounds for use in a method of treating a mammal suffering from or being susceptible to cancer

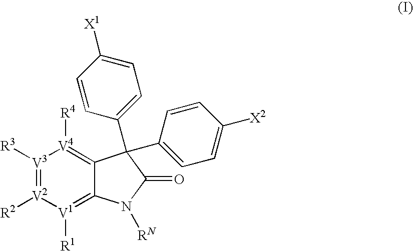

### FIELD OF THE INVENTION

The present invention relates to novel prodrugs of substituted 3,3-diphenyl-1,3-dihydro-indol-2-one compounds.

### BACKGROUND OF THE INVENTION

U.S. Pat. No. 1,624,675 describes O—O-diacyl derivatives of diphenolisatine and that these compounds possess laxative properties.

US 2004/0242563 A1 discloses substituted diphenyl indanone, indane and indole compounds and analogues thereof useful for the treatment or prevention of diseases characterized by abnormal cell proliferation.

WO 05/07107 A1 discloses diphenyl ox-indol-2-one compounds and their use in the treatment of cancer. It is generally suggested that the compounds may be present as prodrugs.

However, there is still a need for improved prodrugs of diphenyl ox-indol-2-one-type compounds.

### BRIEF DESCRIPTION OF THE INVENTION

Following further developments within the field of diphenyl ox-indol-2-one compounds, the present inventors have now found that the 4-position of the phenyl moieties and the N-position of the indole represent very useful handles for the introduction of prodrug groups and that particular prodrug groups, in particular those comprising an amino acid moiety, are particularly promising.

Hence, the present invention provides compounds of the general formulae (I) and (Ia) and (Ib), cf. claims 1, 25, 28 and 29.

The present invention further provides a pharmaceutical composition, cf. claim 35, the utilization of compounds of the general formulae (I) and (Ia) and (Ib) in medicine, cf. claims 37, 38 and 40.

### DETAILED DESCRIPTION OF THE INVENTION

The Compounds of the General Formula (I)

The present invention i.a. relates to particular prodrug compounds which are useful for the treatment of cancer in a mammal.

The useful prodrug compounds have the general formula (I), namely

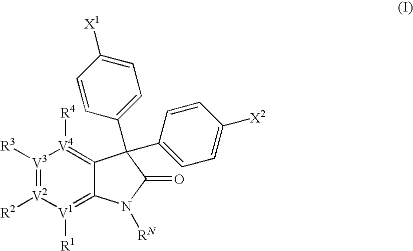

wherein

each of X¹ and X² independently represents a prodrug group of any of the types (i)-(vi)

(i) —O—C(═O)—Z, wherein Z is selected from substituted C₁₋₆-alkyl and —CH(R⁶)N(R⁷)R⁸;

(ii) —O—C(═O)—O—Y, wherein Y is selected from optionally substituted C₁₋₆-alkyl, or —O—Y represents

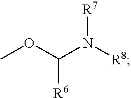

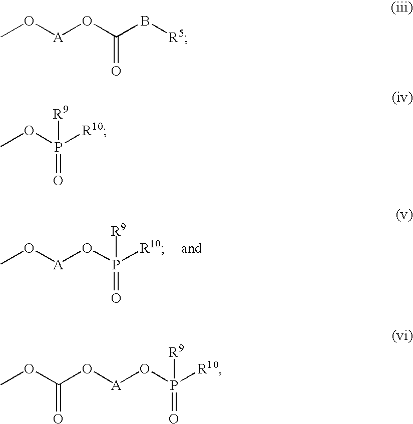

wherein A is selected from optionally substituted C₁₋₆-alkylidene and optionally substituted benzylidene,
B is selected from a single bond, —O— and —S—,
R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug groups (iii) and (vii) may represent an optionally N-substituted amino acid;
R⁶ is selected from hydrogen, optionally substituted C₁₋₆-alkyl, optionally substituted C₂₋₆-alkenyl, aryl, heterocyclyl, and heteroaryl, wherein any aryl, heterocyclyl and heteroaryl may be optionally substituted;
R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R⁷ and R⁸ together with the nitrogen atoms to which they are attached form a heterocyclic ring; and
R⁹ is selected from hydrogen, hydroxy, optionally substituted C₁₋₆-alkyl, optionally substituted C₁₋₆-alkoxy, and optionally substituted C₂₋₆-alkenyloxy;
R¹⁰ is selected from hydroxy, optionally substituted C₁₋₆-alkyl, optionally substituted C₁₋₆-alkoxy, optionally substituted C₂₋₆-alkenyloxy; aryloxy, heterocyclyloxy, and heteroaryloxy, wherein any aryl, heterocyclyl and heteroaryl may be optionally substituted;

provided that R⁹ and R¹⁰ are not both selected from hydroxy and C₁₋₆-alkoxy;

or is selected from hydrogen, hydroxy, optionally substituted C₁₋₆ alkoxy, optionally substituted C₁₋₆alkyl, optionally substituted C₂₋₆alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonyl-amino, C₁₋₆alkylaminocarbonyl or halogen(s) and wherein any aryl, heterocyclyl and heteroaryl may be optionally substituted;

R^{N} represents a prodrug group of any of the types (vii)-(viii)

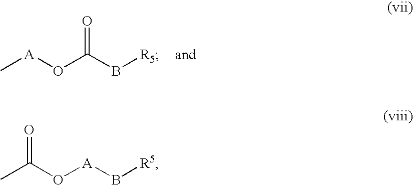

wherein A, B and R⁵ are as defined above for prodrug group (iii);

or is selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);

with the proviso that the compound comprises at least one of the prodrug groups (i)-(viii);

V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;

R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

or R¹ and R² together with the carbon atoms to which they are attached form a ring, e.g. an aromatic ring, a carbocyclic ring, a heterocyclic ring or a heteroaromatic ring, in particular an aromatic ring, a heterocyclic ring or a heteroaromatic ring;

with the proviso that when each of V¹, V², V³ and V⁴ represents a carbon atom, then R^{N}, R¹, R², R³, and R⁴ are not all hydrogen; and

pharmaceutically acceptable salts thereof.

Definitions

In the present context, the term “C₁₋₆-alkyl” is intended to mean a linear, cyclic or branched hydrocarbon group having 1 to 6 carbon atoms, such as methyl, ethyl, propyl, iso-propyl, pentyl, cyclopentyl, hexyl, cyclohexyl, and the term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Similarly, the term “C₂₋₆-alkenyl” is intended to cover linear, cyclic or branched hydrocarbon groups having 2 to 6 carbon atoms and comprising one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

In the present context, i.e. in connection with the terms “alkyl”, “alkylidene”, “alkoxy”, “alkenyl” and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s). Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkylsulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl. In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “benzylidene”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), carboxy, C₁₋₆-alkoxycarbonyl, C₁₋₆-alkylcarbonyl, formyl, aryl, aryl-oxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The expression “optionally N-substituted amino acid” refers to an amino acid moiety wherein the α-nitrogen is represented by —N(R⁷)R⁸, wherein R⁷ and R⁸ are as defined herein. A non-substituted variant is the one where R⁷ and R⁸ are both hydrogen.

The term “prodrug” used herein is intended to mean a compound which—upon exposure to physiological conditions—will liberate a derivative said compound which then will be able to exhibit the desired biological action.

The term “pharmaceutically acceptable salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids. Examples of basic salts are salts where the (remaining) counter ion is selected from alkali metals, such as sodium and potassium, alkaline earth metals, such as calcium, and ammonium ions (⁺N(R)₃R′, where R and R′ independently designates optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

Moreover, it should be understood that the compounds may be present as enantiomers or diastereomers, e.g. when X¹ and X² are different. The present invention encompasses each and every of such possible enantiomers and diastereomers as well as racemates and mixtures enriched with respect to one or the possible enantiomers or diastereomers.

### Embodiments

It should be understood that the compound of the general formula (I) must include at least one prodrug group of any of the types (i), (ii), (iii), (iv), (v), (vi), (vii) and (viii). The compound may comprise only one prodrug group, i.e. one of X¹ and X² is a prodrug group of any of the types (i)-(vi), or R^{N} is a prodrug group of any of the types (vii)-(viii). Alternatively, the compound may comprise more than one prodrug group, e.g. both of X¹ and X² are prodrug group of any of the types (i)-(vi), or R^{N} is a prodrug group of any of the types (vii)-(viii) while one of X¹ and X² is a prodrug group of any of the types (i)-(vi), or both of X¹ and X² are prodrug group of any of the types (i)-(vi) and R^{N} is a prodrug group of any of the types (vii)-(viii).

In the currently most interesting embodiment, at least one of X¹ and X² represents a prodrug group (i) —O—C(═O)—Z, wherein Z is selected from substituted C₁₋₆-alkyl and —CH(R⁶)N(R⁷)R⁸.

In one variant hereof, Z represents a substituted C₁₋₆-alkyl.

In another currently more preferred variant, Z represents —CH(R⁶)—N(R⁷)R⁸.

Typically, R⁶ is selected from hydrogen, optionally substituted C₁₋₆-alkyl, optionally substituted C₂₋₆-alkenyl, aryl, heterocyclyl, and heteroaryl, wherein any aryl, heterocyclyl and heteroaryl may be optionally substituted, or R⁶ and R⁸ together with the intervening carbon and nitrogen atoms to which they are attached form a heterocyclic ring.

Also typically, R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or R⁷ and R⁸ together with the nitrogen atoms to which they are attached form a heterocyclic ring.

Within this variant, however, R⁶ is preferably selected from side chains of essential amino acids, or R⁶ and R⁸ together with the intervening carbon and nitrogen atoms to which they are attached form a heterocyclic ring. In such instances, R⁶ is preferably selected from hydrogen (representing glycine), methyl(alanine), 2-propyl(valine), 2-methyl-1-propyl(leucine), 2-butyl(isoleucine), methylthioethyl(methionine), benzyl(phenylalanine), 3-indolylmethyl(tryptophan), hydroxymethyl(serine), 1-hydroxyethyl(threonine), mercaptomethyl(cysteine), 4-hydroxybenzyl(tyrosine), aminocarbonylmethyl(asparagine), 2-aminocarbonylethyl(glutamine), carboxymethyl(aspartic acid), 2-carboxyethyl(glutamic acid), 4-amino-1-butyl(lysine), 3-guanidino-1-propyl(arginine), and 4-imidazolylmethyl(histidine), or R⁶ and R⁸ together with the intervening carbon and nitrogen atoms to which they are attached form a pyrrolidine ring (proline).

In another preferred embodiment, at least one of X¹ and X² represents a prodrug group (ii) —O—C(═O)—O—Y, wherein Y is selected from optionally substituted C₁₋₆-alkyl, or —O—Y represents

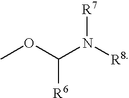

Also preferred are the variants of the above-mentioned embodiment (prodrug groups (i) and (ii)) where R⁷ is hydrogen and R⁸ is selected from hydrogen, optionally substituted C₁₋₆-alkyl, optionally substituted C₁₋₆-alkoxycarbonyl, optionally substituted C₁₋₆-alkylcarbonyl, mono- and di(C₁₋₆-alkyl)aminocarbonyl, C₁₋₆-alkylsulphonyl, C₁₋₆-alkylsulphinyl, aryl, arylcarbonyl, heterocyclyl, heterocyclyloxy, heteroaryl, and heteroaryloxy; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or R⁷ and R⁸ together with the nitrogen atoms to which they are attached form a heterocyclic ring.

In another preferred embodiment, at least one of X¹ and X² represents a prodrug group (iii)

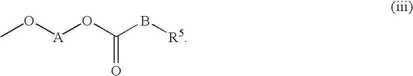

Typically, A is selected from optionally substituted C₁₋₆-alkylidene and optionally substituted benzylidene; B is selected from a single bond, —O— and —S—; and R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug group (iii) may represent an optionally N-substituted amino acid.

In one variant, R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted.

In another currently preferred variant, —C(═O)—B—R⁵ in prodrug group (iii) may represent an optionally N-substituted amino acid, i.e. B is a single bond and R⁵ represents —CH(R⁶)—N(R⁷)R⁸, wherein R⁶, R⁷ and R⁸ are as defined above for prodrug groups (i) and (ii). In particular, R⁶ is preferably selected from side chains of essential amino acids, or R⁶ and R⁸ together with the intervening carbon and nitrogen atoms to which they are attached form a pyrrolidine ring (see the examples above for prodrug groups (i) and (ii)).

In still another embodiment, at least one of X¹ and X² represents a prodrug group of any of the types (iv)-(vi)

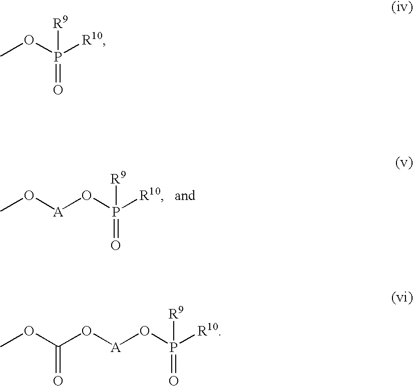

Typically, A is selected from optionally substituted C₁₋₆-alkylidene and optionally substituted benzylidene; R⁹ is selected from hydrogen, hydroxy, optionally substituted C₁₋₆-alkyl, optionally substituted C₁₋₆-alkoxy, and optionally substituted C₂₋₆-alkenyloxy; and R¹⁰ is selected from hydroxy, optionally substituted C₁₋₆-alkyl, optionally substituted C₁₋₆-alkoxy, optionally substituted C₂₋₆-alkenyloxy; aryloxy, heterocyclyloxy, and heteroaryloxy, wherein any aryl, heterocyclyl and heteroaryl may be optionally substituted; provided that R⁹ and R¹⁰ are not both selected from hydroxy and C₁₋₆-alkoxy.

Within this embodiment, R⁹ is preferably selected from hydrogen and hydroxy, and R¹⁰ is preferably selected from optionally substituted C₁₋₆-alkoxy, optionally substituted C₂₋₆-alkenyloxy; aryloxy, heterocyclyloxy, and heteroaryloxy, wherein any aryl, heterocyclyl and heteroaryl may be optionally substituted. More particular, R⁹ is hydroxy, and R¹⁰ is selected from optionally substituted C₁₋₆-alkoxy, aryloxy, heterocyclyloxy, and heteroaryloxy, wherein any aryl, heterocyclyl and heteroaryl may be optionally substituted.

In yet another embodiment, R^{N} represents a prodrug group or any of the types (vii)-(viii)

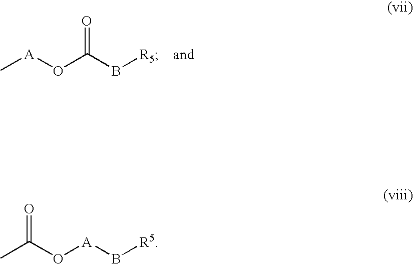

Typically, A is selected from optionally substituted C₁₋₆-alkylidene and optionally substituted benzylidene; B is selected from a single bond, —O—, and —S—; and R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug group (vii) may represent an optionally N-substituted amino acid.

In a currently preferred variant, —C(═O)—B—R⁵ in prodrug group (vii) represent an optionally N-substituted amino acid, i.e. B is a single bond and R⁵ represents —CH(R⁶)—N(R⁷)R⁸, wherein R⁶, R⁷ and R⁸ are as defined above for prodrug groups (i) and (ii). In particular, R⁶ is preferably selected from side chains of essential amino acids, or R⁶ and R⁸ together with the intervening carbon and nitrogen atoms to which they are attached form a pyrrolidine ring (see the examples above for prodrug groups (i) and (ii)).

The function of V¹, V², V³, and V⁴ is mainly believed to be of sterical character, i.e. determinative for the orientation of the groups R¹-R⁴. It is, however, also believed that the selection of a heteroatom as one or more of V¹, V², V³, and V⁴ may create dipole interactions with other entities and thereby have influence on, e.g., the solubility of the compounds of the general formula (I).

V¹, V², V³, and V⁴ are independently selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring. Particularly useful examples of such aromatic rings and heteroaromatic rings are those selected from a benzene ring, a thiophene ring (V¹═S, V²═V³═C(−) and V⁴=bond; V²═S, V¹═V³═C(−) and V⁴=bond; or V³═S, V¹═V²═C(−) and V⁴=bond), a furan ring (V¹═O, V²═V³═C(−) and V⁴=bond; V²═O, V¹═V³═C(−) and V⁴=bond; or V³═O, V¹═V²═C(−) and V⁴=bond), a pyrazole ring (V¹═N(−), V²═N, V³═C(−) and V⁴=bond; V¹═N, V²═N(−), V³═C(−) and V⁴=bond), an imidazole ring (V¹═N(−), V²═C(−), V³═N and V⁴=bond; V¹═N, V²═C(−), V³═N(−) and V⁴=bond), a pyridine ring (V¹═N, V²═V³═V⁴═C(−); V²═N, V¹═V³═V⁴═C(−); V³═N, V¹═V²═V⁴═C(−) and V⁴═N, V¹═V²═V³═C(−)), a pyrimidine ring (V¹═V³═N, V²═V⁴═C(−); V²═V⁴═N, V¹═V³═C(−)), pyrazines (V¹═V⁴═N, V²═V³═C(−)), a pyridazine ring (V¹═V²═N, V³═V⁴═C(−); V²═V³═N, V¹═V⁴═C(−); V³═V⁴═N, V¹═V²═C(−)), a thiazole ring (V¹═N, V²═C(−), V³═S, V⁴=bond; V¹═S, V²═C(−), V³═N, V⁴=bond), and an isothiazole ring (V¹═N, V²═S, V³═C(−), V⁴=bond; V¹═S, V²═N, V³═C(−), V⁴=bond; V¹═C(−), V²═S, V³═N, V⁴=bond; V¹═C(−), V²═N, V³═S, V⁴=bond).

The meaning of V¹, V², V³ and V⁴ for each heteroaromatic ring is merely specified for the purpose of illustrating that various orientations of the heteroatoms are possible. Furthermore, it should be understood that the respective rings carry the substituents R¹, R², R³ and R⁴ (where applicable) in accordance with the general formula (I). Thus, specification of “C(−)” and “N(−)” as possible meanings of V¹, V², V³ and V⁴ is made for the purpose of describing that the atoms in question carry a substituent (which may be hydrogen). Specification of “N” means that the respective atoms do not carry an “R” substituent, i.e. the corresponding “R” substituent is absent.

In one embodiment, —V¹—V²—V³—V⁴— together with the atoms to which V¹ and V⁴ are attached form a ring selected from a benzene ring, a thiophene ring, a furan ring, a pyrazole ring, an imidazole ring, a pyridine ring, a pyrimidine ring, pyrazines, and a pyridazine ring, in particular from a benzene ring and a pyridine ring where the nitrogen atom represents V³ (see also the Examples). In accordance with the general formula (I), the respective ring (aromatic or heteroaromatic) carries the substituents R¹-R⁴ (where applicable).

The substituents R¹-R⁴ (where applicable) are believed to be at least partly responsible for the biological effect, e.g. the ability of the compounds to inhibit cell proliferation in cancer cells.

In one embodiment, R¹, R², R³, and R⁴ are, when attached to a carbon atom, independently selected from hydrogen, optionally substituted C₁₋₆-alkyl, optionally substituted C₂₋₆-alkenyl, hydroxy, optionally substituted C₁₋₆-alkoxy,optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and R¹, R², R³, and R⁴ are, when attached to a nitrogen atom, independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted.

More particularly, R¹, R², R³, and R⁴ are independently selected from hydrogen, halogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkylcarbonylamino, C₁₋₆-alkylsulphonylamino, mono- and di(C₁₋₆-alkyl)aminosulfonyl, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), such as from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkyl-carbonylamino, C₁₋₆-alkylsulphonylamino, mono- and di(C₁₋₆-alkyl)aminosulfonyl, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

As an alternative to the above, R¹ and R² may in one embodiment together with the carbon atoms to which they are attached form a heterocyclic ring or a heteroaromatic ring; and in another embodiment, R¹ and R² may together with the carbon atoms to which they are attached form an aromatic ring or a carbocyclic ring.

Preferably, R¹, R², R³ and R⁴ are not all hydrogen.

In a currently highly preferred embodiment, R¹ and R² are both halogen, in particular, R¹ and R² are both fluoro. In a variant within this embodiment, R^{N}, R³ and R⁴ are all hydrogen.

It is believed that R^{N} may be selected from a wide variety of substituents including the prodrug group (vi). If not being a prodrug group, R^{N} may advantageous be selected from hydrogen, C₁₋₆-alkyl, amino, and C₁₋₆-alkylcarbonylamino. Most preferred is the variants wherein R^{N} is selected from hydrogen and C₁₋₆-alkyl, in particular from hydrogen and methyl, most typical hydrogen.

In one currently preferred variant,

each of V¹, V², V³, and V⁴ represents a carbon atom;

R¹ and R² are both fluoro;

R³ and R⁴ are all hydrogen;

R^{N} is hydrogen;

at least one of X¹ and X² represents a prodrug group (i) —O—C(═O)—CH(R⁶)—N(R⁷)R⁸,

wherein R⁶ is selected from hydrogen(glycine), methyl(alanine), 2-propyl(valine), 2-methyl-1-propyl(leucine), 2-butyl(isoleucine), methylthioethyl(methionine), benzyl(phenylalanine), 3-indolylmethyl(tryptophan), hydroxymethyl(serine), 1-hydroxyethyl(threonine), mercaptomethyl(cysteine), 4-hydroxybenzyl(tyrosine), aminocarbonylmethyl(asparagine), 2-aminocarbonylethyl(glutamine), carboxymethyl(aspartic acid), 2-carboxyethyl(glutamic acid), 4-amino-1-butyl(lysine), 3-guanidino-1-propyl(arginine), and 4-imidazolylmethyl(histidine), or R⁶ and R⁸ together with the intervening carbon and nitrogen atoms to which they are attached form a pyrrolidine ring (proline);

R⁷ is hydrogen and R⁸ is selected from hydrogen, optionally substituted C₁₋₆-alkyl, optionally substituted C₁₋₆-alkoxycarbonyl, optionally substituted C₁₋₆-alkylcarbonyl, mono- and di(C₁₋₆-alkyl)aminocarbonyl, C₁₋₆-alkylsulphonyl, C₁₋₆-alkylsulphinyl, aryl, arylcarbonyl, heterocyclyl, heterocyclyloxy, heteroaryl, and heteroaryloxy; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or R⁷ and R⁸ together with the nitrogen atoms to which they are attached form a heterocyclic ring; and

any other of X¹ or X² is selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted.

In one particular embodiment, R¹ is selected from hydrogen, halogen, C₁₋₆-alkyl, trifluoromethyl and C₁₋₆-alkoxy, when V¹ is a carbon atom.

In a further embodiment, R² is selected from hydrogen, halogen, optionally substituted aryl, optionally substituted aryloxy, and optionally substituted heteroaryl, when V² is a carbon atom.

In a still further embodiment, R³ is selected from hydrogen, optionally substituted C₁₋₆-alkoxy, halogen, cyano, optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl, when V³ is a carbon atom.

In an even still further embodiment, R⁴ is hydrogen, when V⁴ is a carbon atom.

The Compounds of General Formula (Ia)

It has been found that the compounds wherein at least one prodrug group comprising an amino acid moiety is present represents a particularly interesting aspect of the present invention. Hence, the present invention also provides a compound of the general formula (Ia)

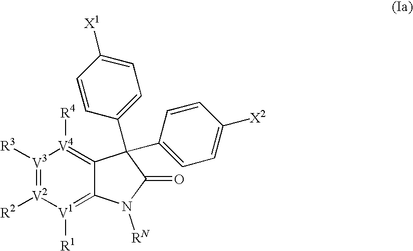

wherein

at least one of X¹, X² and R^{N} represent a prodrug group comprising an amino acid moiety,

any of X¹ and X² not being a prodrug group independently being selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted;

and R^{N} not being a prodrug group being selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);

V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;

R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

or R¹ and R² together with the carbon atoms to which they are attached form a ring, e.g. an aromatic ring, a carbocyclic ring, a heterocyclic ring or a heteroaromatic ring, in particular an aromatic ring, a heterocyclic ring or a heteroaromatic ring;

with the proviso that when each of V¹, V², V³ and V⁴ represents a carbon atom, then R^{N}, R¹, R², R³, and R⁴ are not all hydrogen; and

pharmaceutically acceptable salts thereof.

In this embodiment, at least one of X¹ and X² is preferably a prodrug group of any of the types (ia) and (iiia)

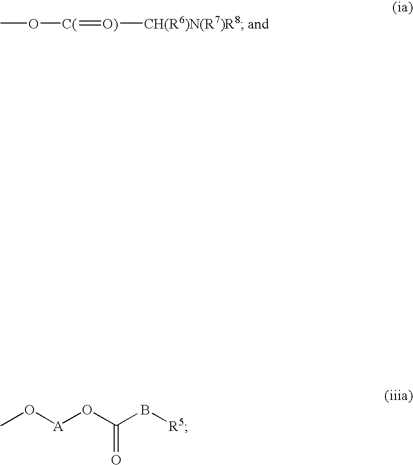

wherein A is selected from optionally substituted C₁₋₆-alkylidene and optionally substituted benzylidene,

—C(═O)—B—R⁵ represent an optionally N-substituted amino acid;

R⁶ is selected from hydrogen(glycine), methyl(alanine), 2-propyl(valine), 2-methyl-1-propyl(leucine), 2-butyl(isoleucine), methylthioethyl(methionine), benzyl(phenylalanine), 3-indolylmethyl(tryptophan), hydroxymethyl(serine), 1-hydroxyethyl(threonine), mercaptomethyl(cysteine), 4-hydroxybenzyl(tyrosine), aminocarbonylmethyl(asparagine), 2-aminocarbonylethyl(glutamine), carboxymethyl(aspartic acid), 2-carboxyethyl(glutamic acid), 4-amino-1-butyl(lysine), 3-guanidino-1-propyl(arginine), and 4-imidazolylmethyl(histidine), or R⁶ and R⁸ together with the intervening carbon and nitrogen atoms to which they are attached form a pyrrolidine ring (proline);

R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or R⁷ and R⁸ together with the nitrogen atoms to which they are attached form a heterocyclic ring.

Moreover, R^{N} may (as the sole prodrug group or in combination with another prodrug group as X¹ and/or X²) represents a prodrug group of the type (vii)

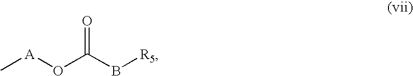

wherein A is selected from optionally substituted C₁₋₆-alkylidene and optionally substituted benzylidene, and —C(═O)—B—R⁵ represent an optionally N-substituted amino acid.

The further specifications and indications of preferred meanings for the substituents X¹, X², R^{N}, V¹, V², V³, V⁴, R¹, R², R³, R⁴, R⁵, R⁶, R⁷, and R⁸ given in connection with compound of general formula (I) also apply for the compounds of general formula (Ia), mutatis mutandis.

The Compounds of General Formula (Ib)

It has also been found that compounds comprising other particular prodrug groups represents a further particularly interesting aspect of the present invention. Hence, the present invention also provides a compound of the general formula (Ib)

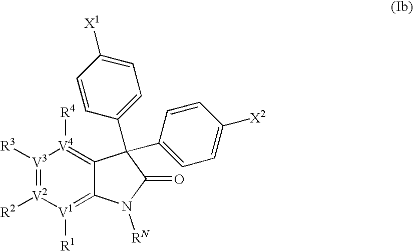

wherein

each of X¹ and X² independently represents a prodrug group of any of the types (ix)-(x)

(ix) —O—C(═O)—Z, wherein Z is selected from optionally substituted C₁₋₆-alkenyl and —N(R⁷)R⁸; and

(x) —O—CH₂—C(═O)—Y, wherein Y is selected from optionally substituted C₁₋₆-alkyl;
wherein R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted, or R⁷ and R⁸ together with the nitrogen atoms to which they are attached form an optionally substituted heterocyclic ring;

or is selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted;

R^{N} represents a prodrug group of any of the types (xi)-(xii)

(xi) -A-C(═O)—B—R⁵ ; and

(xii) —(CH₂—CH₂—O)₁₋₁₀—R⁵,
wherein A is selected from optionally substituted C₁₋₆-alkylidene and optionally substituted benzylidene,
B is selected from a single bond, —O— and —S—, and
R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug group (xi) may represent an optionally N-substituted amino acid;

or is selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);

with the proviso that the compound comprises at least one of the prodrug groups (ix)-(xii);

V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;

R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;

or R¹ and R² together with the carbon atoms to which they are attached form a ring, e.g. an aromatic ring, a carbocyclic ring, a heterocyclic ring or a heteroaromatic ring, in particular an aromatic ring, a heterocyclic ring or a heteroaromatic ring;

with the proviso that when each of V¹, V², V³ and V⁴ represents a carbon atom, then R^{N}, R¹, R², R³, and R⁴ are not all hydrogen; and

pharmaceutically acceptable salts thereof.

In one variant, at least one of X¹ and X² represents a prodrug group (ix) —O—C(═O)—Z.

In another variant, at least one of, wherein X¹ and X² represents a prodrug group (x) —O—CH₂—C(═O)—Y.

In a further variant, R^{N} represents a prodrug group (xi) -A-C(═O)—B—R⁵.

In a still further variant, R^{N} represents a prodrug group (xii) —(CH₂—CH₂—O)₁₋₁₀—R⁵.

The further specifications and indications of preferred meanings for the substituents X¹, X², R^{N}, V¹, V², V³, V⁴, R¹, R², R³, R⁴, R⁵, R⁷, and R⁸ given in connection with compound of general formula (I) also apply for the compounds of general formula (Ib), mutatis mutandis.

Currently Most Preferred Compounds

Presently very interesting compounds of the formulae (I) and (Ia) and (Ib) are those listed in the following:

(2S)-4-(6,7-difluoro-3-(4-methoxyphenyl)-2-oxoindolin-3-yl)phenyl 2-aminopropanoate hydrochloride,

(2S,2′S)-4,4′-(6,7-difluoro-2-oxoindoline-3,3-diyl)bis(4,1-phenylene)bis(2-aminopropanoate dihydrochloride,

4,4═-(6,7-difluoro-2-oxoindoline-3,3-diyl)bis(4,1-phenylene)bis(2-aminoacetate)ditrifluoroacetic acid salt,

(2S,2′S)-4,4′-(6,7-difluoro-2-oxoindoline-3,3-diyl)bis(4,1-phenylene)bis(3-methyl-2-(methylamino)butanoate)dihydrochloride,

4,4′-(6,7-difluoro-2-oxoindoline-3,3-diyl)bis(4,1-phenylene)bis(2-dimethylamino)acetate),

(2S)-4-(6,7-difluoro-3-(4-methoxyphenyl)-2-oxoindolin-3-yl)phenyl 2-aminophenylpropanoate hydrochloride,

4-(3-(4-chlorophenyl)-6,7-difuoro-2-oxoindolin-3-yl)phenyl(2-morpholinoethoxy)methyl carbonate,

(7-fluoro-3,3-bis(4-hydroxyphenyl)-6-methyl-2-oxoindolin-1-yl)methyl 2-morpholinoethyl carbonate,

(4,4′-(6-fluoro-7-methyl-2-oxoindoline-3,3-diyl)bis(4,1-phenylene)bis(oxy)bis(methylene)bis(2-morpholinoethyl) dicarbonate, and

4-(3-(4-chlorophenyl)-6,7-dimethyl-2-oxoindolin-3-yl)phenyl methyl methylphosphonate

Presently very interesting compounds of the formula (Ib) are those listed in the following:

6,7-Difluoro-3-(4-fluorophenyl)-1-(2-hydroxyethyl)-3-(4-hydroxyphenyl)-1,3-dihydro-2H-indol-2-one,

4-[6,7-Difluoro-3-(4-fluorophenyl)-2-oxo-2,3-dihydro-1H-indol-3-yl]phenyl 3-morpholinopropanoate,

4-[6,7-Difluoro-3-(4-fluorophenyl)-2-oxo-2,3-dihydro-1H-indol-3-yl]phenyl 3-(4-methylpiperazino)propanoate,

[3-(4-{[(Isopropoxycarbonyl)oxy]methoxy}phenyl)-3-(4-methoxyphenyl)-2-oxo-2,3-dihydro-1H-indol-1-yl]methyl isopropyl carbonate,

Benzyl 2-[3-{4-[2-(benzyloxy)-2-oxoethoxy]phenyl}-3-(4-methoxyphenyl)-2-oxo-2,3-dihydro-1H-indol-1-yl]acetate,

4-(3-{4-[(Anilinocarbonyl)oxy]phenyl}-6,7-difluoro-2-oxo-2,3-dihydro-1H-indol-3-yl)phenyl N-phenylcarbamate,

4-[6,7-Difluoro-3-(4-fluorophenyl)-2-oxo-2,3-dihydro-1H-indol-3-yl]phenyl N,N-dimethylcarbamate,

4-[6-Fluoro-3-(4-fluorophenyl)-7-methyl-2-oxo-2,3-dihydro-1H-indol-3-yl]phenyl N,N-dimethylcarbamate, and

4-[6-Fluoro-3-(4-fluorophenyl)-7-methyl-2-oxo-2,3-dihydro-1H-indol-3-yl]phenyl acrylate.

Preparation of Compounds

The compounds of the present invention can be prepared in a number of ways well known to those skilled in the art of organic synthesis. The compounds of the present invention can be synthesized using the methods outline below and in the Examples section, together with methods known in the art of organic synthetic organic chemistry, or variations thereof as appreciated by those skilled in the art. Preferred methods include, but are not limited to, those described below.

The novel compounds of formulae (I) and (Ia) and (Ib) may be prepared using the reactions and techniques described in this section. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature duration of experiment and work-up procedures, are chosen to be conditions of standard for that reaction, which should be readily recognized by one skilled in the art. It is understood by one skilled in the art of organic synthesis that the functionality present on various portions of the educt molecule must be compatible with the reagents and reactions proposed. Not all molecules of formulae (I) and (Ia) and (Ib) falling into a given class may be compatible with some of the reaction conditions required in some of the methods described. Such restrictions to the substituents which are compatible with the reaction conditions will be readily apparent to one skilled in the art and alternative methods can be used.

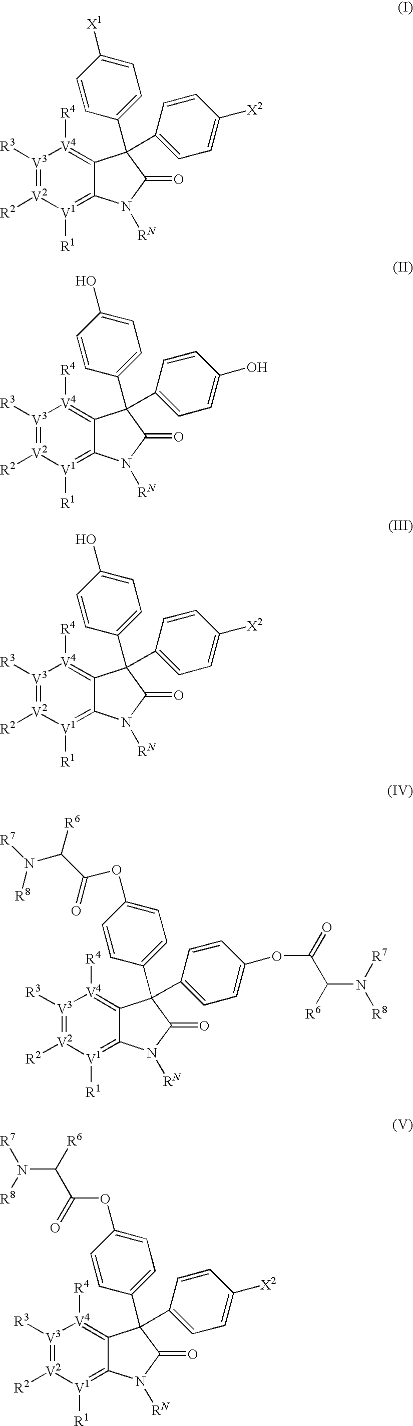

Compounds according to the present invention in which X¹ and/or X² is an amino acid ester may be prepared from compounds of general formula (II) or (III) by coupling with a protected amino acid and subsequent removal of the protecting groups, if any, to yield compounds of general formula (IV) and (V). The condensation is carried out using any of the many methods for the formation of ester bonds known to one skilled in the art of organic synthesis. These methods include, but are not limited to, use of standard coupling procedures such as use of symmetric carbonic anhydrides, mixed carbonic anhydride (e.g. isobutyl chloroformate) method, carbodiimides (e.g. N,N-dimethylaminopropyl-N′-ethyl carbodiimide, dicyclohexyl carbodiimide, diisopropyl carbodiimide), active ester (e.g. pentaflurophenyl ester, p-nitrophenyl ester, N-hydroxysuccinic imido ester) method, carbonyldiimidazole method, azide method, phosphorous reagents such as BOP-Cl, conversion of the protected amino acid derivative into an acid chloride. Some of these methods (especially carbodiimide) can be enhanced by addition of e.g. 1-hydroxybenzotriazole or N,N-dimethylaminopyridine.

Protection groups as referred to above are well known per se, for example from the techniques of peptide chemistry. Amino groups can often be protected by tert-butyloxycarbonyl, benzyloxycarbonyl or acetyl groups, or in the form of a phtalimido group. Hydroxy groups are often protected as readily cleavable ethers such as the t-butyl or benzyl ether, or as readily cleavable esters such as the acetate. Carboxylic acid groups are often protected as readily cleavable esters such as the t-butyl or benzyl ester. Thiols are often protected as readily cleavable ethers such as the trityl ether.

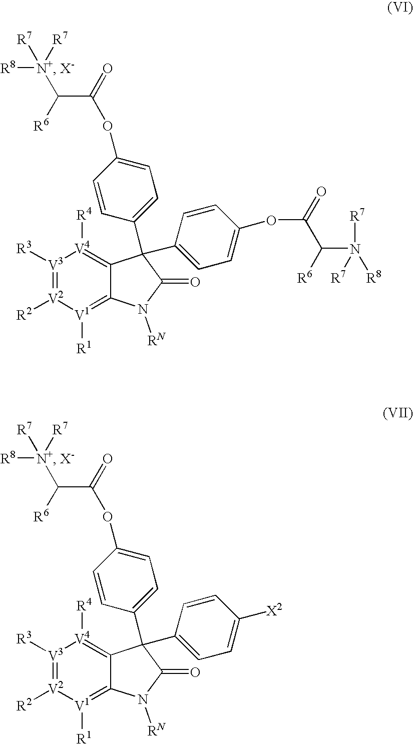

If R⁷ and R⁸ are both alkyl groups compounds of general formula (IV) and (V) can be converted into the corresponding trialkylammonium salts (VI) and (VII), e.g. by reaction with an alkyl halide and a base or methyl methane sulfonate.

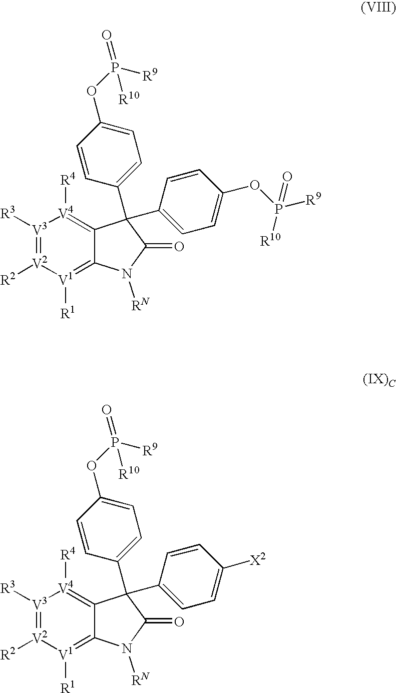

Compounds according to the present invention in which X₁ and/or X₂ is a phosphonate group or a phosphinate group (VIII) and (IX) may be prepared from compounds of general formula (II) or (III) e.g. by condensation with a phosphonochloridate or a phosphinic chloride in the presence of a base.

Compounds according to the present invention in which X¹ and/or X₂ is —O-A-O(C═O)—B—R⁵ (X) and (XI) can be prepared form compounds of general (II) and (III), in which R^{N} is an amide protecting group (e.g. a silyl-type or benzyl type protecting group) by reaction with a base and chloromethyl or iodomethyl esters of general formula (XII), and subsequent removal of the amide protecting group (e.g. by use of fluoride ion or hydrogenation). The chloromethyl or iodomethyl esters of general formula (XII) may be prepared as described in Bioorg. Med. Chem. Lett. (2005) 13 2491-2494.

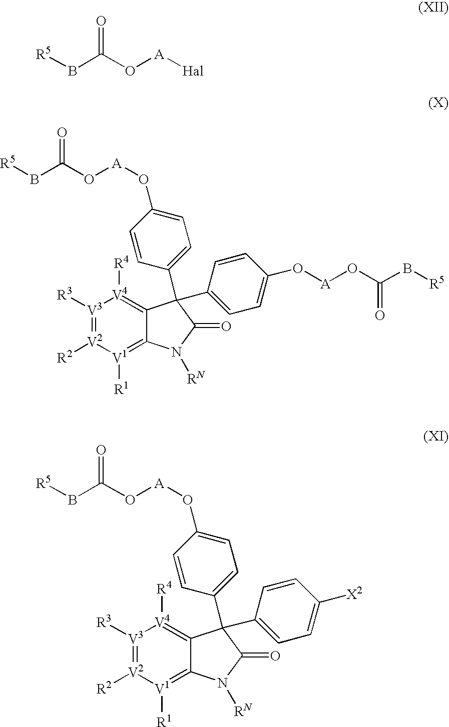

Alternatively, compounds of general formula (X) and (XI) may prepared by similar methods to those described in Bioorg. Med. Chem. Lett. (2003) 1695-1698 after suitable protection of the amide group and subsequent removal of the protecting group, as described above.

Compounds according to the present invention in which R^{N} is —O-A-O(C═O)—B—R⁵, X¹ and/or X₂ is —O-A-O(C═O)—B—R⁵ (X) and (XI) can be prepared form compounds of general (II) and (III) by reaction with a base of the right choice and chloromethyl or iodomethyl esters of general formula (XII).

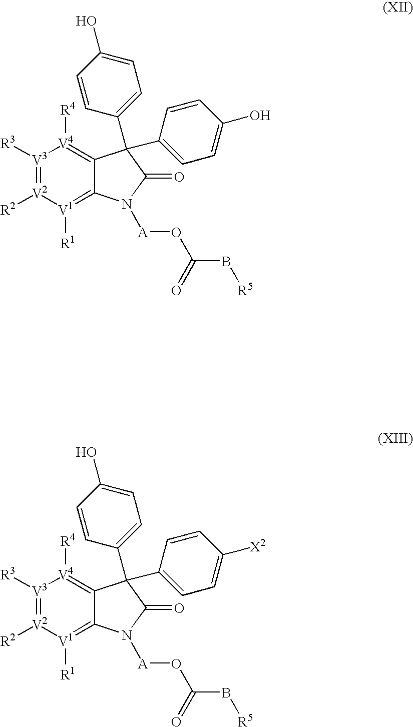

Compounds according to the present invention in which R^{N} is —O-A-O(C═O)—B—R⁵, X₁ and/or X₂ is hydrogen (XII) and (XIII) can be prepared form compounds of general (II) and (III) reaction with a base of the right choice, e.g. NaH, and chloromethyl or iodomethyl esters of general formula (XII). The phenolic group(s) may require protection prior to derivatization of the amide function, e.g. with a Boc-group, a silyl ether or an acyl group, followed by deprotection as the last step.

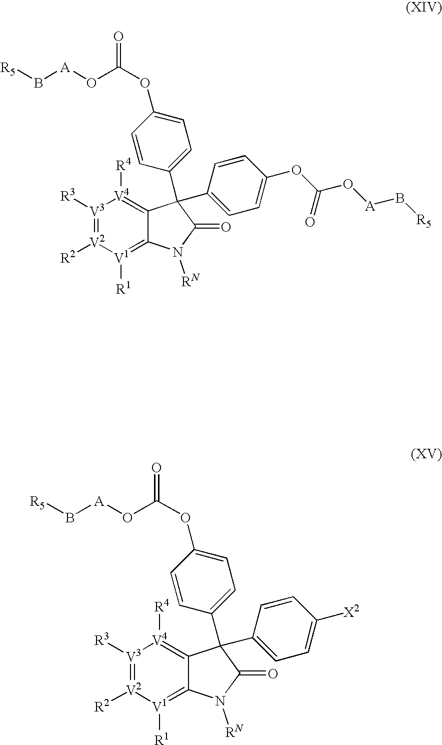

Compounds according to the present invention in which X¹ and/or X₂ is O-A-O(C═O)—B—R⁵ (XIV) and (XV) can be prepared form compounds of general (II) and (III) by reaction with chloromethyl carbonochloridate and a base, e.g. potassium carbonate or cesium carbonate, and subsequent reaction of the resulting chloromethyl phenylcarbonate with an alcohol or a thiol and a base. Compounds according to the present invention in which R^{N} as well as X₁ and/or X₂ is —O—CO—CH₂—OR (XIV) and (XV) can be prepared form compounds of general (II) and (III) can be prepared as described above with a different choice of base, e.g. NaH.

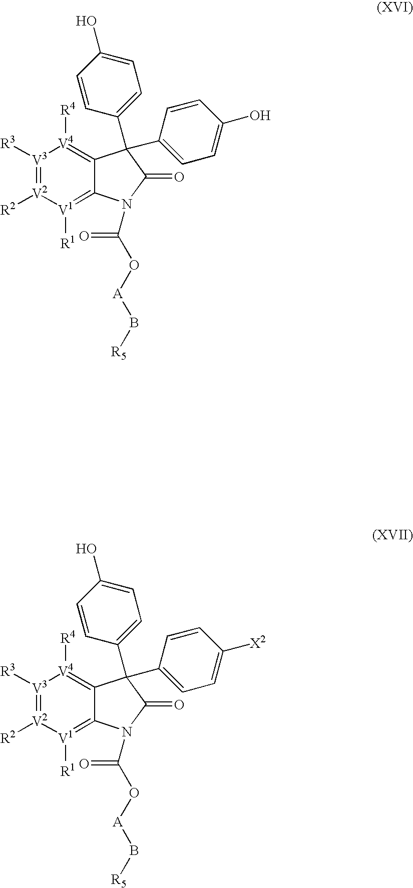

Compounds according to the present invention in which R^{N} is O-A-O(C═O)—B—R⁵ (XVI) and (XVII) can be prepared form compounds of general (II) and (III) by protection of the phenolic function(s), e.g. with a Boc-group, a silyl ether or an acyl group, reaction with chloromethyl carbonochloridate and a base, e.g. NaH, and subsequent reaction of the resulting chloromethyl 3,3-bis(phenyl)-2-oxoindoline-1-carboxylate with an alcohol or a thiol. and a base, followed by removal of the protecting group(s).

Medical Uses

The compounds of the general formulae (I) and (Ia) and (Ib) are believed to be particularly useful in the treatment of cancer. The term cancer is typically describing cell growth not under strict control. In one embodiment of the invention, treatment of cancers in which inhibition of protein synthesis and/or inhibition of activation of the mTOR pathway is an effective method for reducing cell growth. Examples of such cancers are breast cancer, renal cancer, multiple myeloma, leukemia, glio blastoma, rhabdomyosarcoma, prostate, soft tissue sarcoma, colorectal sarcoma, gastric carcinoma, head and neck squamous cell carcinoma, uterine, cervical, melanoma, lymphoma, and pancreatic cancer.

Hence, the present invention generally provides a compound of the general formula (I) or (Ia) or (Ib) as defined herein for use as a medicament; more particular, the use of a compound of the general formula (I) or (Ia) or (Ib) as defined herein for the preparation of a medicament for the treatment of cancer in a mammal. Such medicaments may further comprise one or more other chemotherapeutic agents.

Moreover, the present invention provides a method of treating a mammal suffering from or being susceptible to cancer, the method comprising administering to the mammal a therapeutically effective amount of a compound of the general formula (I) or (Ia) or (Ib) as defined herein.

Formulation of Pharmaceutical Compositions

The compounds of the general formulae (I) and (Ia) and (Ib) are suitably formulated in a pharmaceutical composition so as to suit the desirable route of administration.

The administration route of the compounds may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds may be contained in any appropriate amount in a pharmaceutical composition, and are generally contained in an amount of about 1-95%, e.g. 1-10%, by weight of the total weight of the composition. The composition may be presented in a dosage form which is suitable for the oral, parenteral, rectal, cutaneous, nasal, vaginal and/or ocular administration route.

Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or excipient. Pharmaceutically acceptable carriers or excipients are those known by the person skilled in the art. Formation of suitable salts of the compounds of the Formulae (I) and (Ia) and (Ib) will also be evident in view of the before-mentioned.

Thus, the present invention provides in a further aspect a pharmaceutical composition comprising a compound of the general Formula (I) or (Ia) or (Ib) in combination with a pharmaceutically acceptable carrier.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (saw-tooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye will be well-known to those skilled in the art of pharmaceutical formulation. Specific formulations can be found in “Remington's Pharmaceutical Sciences”.

Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The pharmaceutical compositions may also be emulsions of the compound(s) and a lipid forming a micellular emulsion.

For parenteral, subcutaneous, intradermal or topical administration the pharmaceutical composition may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

Dosages

In one embodiment, the pharmaceutical composition is in unit dosage form. In such embodiments, each unit dosage form typically comprises 0.1-500 mg, such as 0.1-200 mg, e.g. 0.1-100 mg, of the compound.

More generally, the compound are preferably administered in an amount of about 0.1-250 mg per kg body weight per day, such as about 0.5-100 mg per kg body weight per day.

For compositions adapted for oral administration for systemic use, the dosage is normally 0.5 mg to 1 g per dose administered 1-4 times daily for 1 week to 12 months depending on the disease to be treated.

The dosage for oral administration of the composition in order to prevent diseases or conditions is normally 1 mg to 100 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 100 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 50 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

Combination Treatment

In an intriguing embodiment of the present invention, the compound of the general formula (I) or (Ia) or (Ib) is used therapeutically in combination with one or more other chemotherapeutic agents. Examples of such chemotherapeutic agents are those selected from daunorubicin, docetaxel, prednisone, dexamethasone, decadron, altretamine, amifostine, aminoglutethimide, dactinomycin, anastrozole, asparaginase, bicalutamide, bleomycin, busulfan, carboplatin, carmustine, chlorambucil, chlorodeoxyadenosine, cisplatin, cytosine arabinoside, dacarbazine, doxorubicin, epirubicin, estramustine, diethylstilbestrol, fludarabine, flutamide, 5-fluorouracil, gemcitabine, goserelin, idarubicin, irinotecan, levamisole, lomustine, mechlorathamine, alkeran, mercaptopurine, taxol (e.g. paclitaxel). In particular, the further chemotherapeutic agent is selected from taxanes such as Taxol, Paclitaxel and Docetaxel.

Thus, with respect to the use and the method of treatment defined herein, the medicament may further comprise one or more other chemotherapeutic agents.

With respect to the pharmaceutical composition defined herein, such a composition may further comprise one or more other chemotherapeutic agents.

### Examples

For nuclear magnetic resonance ¹H NMR spectra (300 MHz) and ¹³C NMR (75.6) chemical shift values (δ) (in ppm) are quoted, unless otherwise specified, for deuteriochloroform solutions relative to tetramethylsilane (δ=0.0) or chloroform (δ=7.25) or deuteriochloroform (δ=76.81 for ¹³C NMR) standards. The value of a multiplet, either defined (dublet (d), triplet (t) quartet (q)) or not (m) at he approximate mid point is given unless a range is quoted. (bs) indicates a broad singlet. The organic solvents used were anhydrous.

The following abbreviations have been used throughout:

BOC tert-butyloxycarbonyl

DCM dichloromethane

DIC diisoproylcarbodiimide

DMAP dimethylaminopryridine

DMF N,N-dimethylformamide

EDC N-(dimethylaminopropyl)-N′-ethylcarbodiimide

EtOAc ethyl acetate

NMR nuclear magnetic resonance

rt room temperature

TFA trifluoro acetic acid

TLC thin layer chromatography

Starting materials can be prepared as described in the literature, e.g. in WO 2005/097107.

General Procedure 1: Formation of BOC-Protected Amino Acid Ester Derivatives from Phenols of General Formula (II).

The BOC-protected amino acid (12.0 mmol) was dissolved in DCM (20 mL), cooled in an ice bath under argon and DIC (6.0 mmol) was added drop-wise with stirring. After 1½ h the mixture was filtered, concentrated, redissolved in pyridine (10 mL) and the phenol of general formula (II) (2.0 mmol) was added with stirring. The mixture was stirred at room temperature overnight, concentrated three times with toluene. The residue was purified by chromatography (1% methanol in DCM) or crystallization to afford BOC-protected amino acid ester derivatives from phenols of general formula (II).

General Procedure 2: Formation of BOC-Protected Amino Acid Ester Derivatives from Phenols of General Formula (III).

The phenol of general formula (III) (0.42 mmol) and the BOC-protected amino acid (0.59 mmol) were dissolved in DCM, EDC (0.71 mmol) and DMAP (10 mg) were added with stirring and the mixture was left at room temperature overnight, transferred to a separatory funnel with EtOAc/H₂O and shaken. The aqueous phase was extracted twice more with EtOAc. The combined organic layers were washed with brine, dried (MgSO₄) and concentrated. The residue was purified by chromatography (1% methanol in DCM or mixtures of petroleum ether and EtOAc) to afford BOC-protected amino acid ester derivatives from phenols of general formula (III).

General Procedure 3: Removal of BOC-Protecting Group from BOC-Protected Amino Acid Ester Derivatives of Phenols of General Formula (II) or General Formula (III) Using HCl to Yield Compounds of General Formula (IV) or (V).

The BOC-protected amino acid ester derivative of phenol of general formula (II) or (III)) was dissolved in DCM or methanol and HCl in diethyl ether (1 M) was added. When TLC showed no remaining starting material the mixture was concentrated and if necessary purified by crystallization to afford compounds of general formula (IV) or (V).

General Procedure 4: Removal of BOC-Protecting Group from BOC-Protected Amino Acid Ester Derivatives of Phenols of General Formula (II) or General Formula (III) Using TFA to Yield Compounds of General Formula (IV) or (V).

The BOC-protected amino acid ester derivative of phenol of general formula (II) or (III)) was dissolved in TFA. After 30 minutes the mixture was concentrated twice with toluene and if necessary purified by crystallization to afford compounds of general formula (IV) or (V).

General Procedure 5: Formation of N,N-Dimethylamino Acid Esterderivatives from Phenols of General Formula (II) or (III).

The phenol of general formula (II) or (III) (0.27 mmol) and the N,N-dimethylamino acid (0.78 mmol) were dissolved in DMF, EDC (0.88 mmol) and DMAP (0.27 mmol) were added with stirring. The mixture was stirred at room temperature overnight, concentrated, extracted with EtOAc/H₂O. The aqueous layer was back-extracted twice with EtOAc, and the combined organic layers were washed with brine, dried (MgSO₄) and concentrated. The residue was purified by crystallization to afford compound of general formula (IV) or (V).

### Preparation 1: (2S)-4-(6,7-difluoro-3-(4-methoxyphenyl)-2-oxoindolin-3-yl)phenyl 2-(tert-butoxycarbonylamino)propanoate (Compound 1)

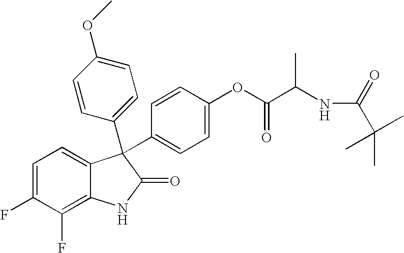

General procedure 2. Starting materials: 6,7-difluoro-3-(4-hydroxy-phenyl)-3-(4-methoxy-phenyl)-1,3-dihydro-indol-2-one and N-BOC-L-alanine. ¹H-NMR (CDCl₃) δ 8.95 (bs, 1H), 7.27 (d, 2H), 7.13 (d, 2H), 7.03 (d, 2H), 6.95-6.75 (m, 4H), 5.17 (d, 1H), 4.56 (q, 1H), 3.80 (s, 3H), 1.53 (d, 3H), 1.45 (s, 9H).

### Preparation 2: (2S)-4-(6,7-difluoro-3-(4-methoxyphenyl)-2-oxoindolin-3-yl)phenyl 2-(tert-butoxycarbonylamino)3-phenylpropanoate (Compound 2)

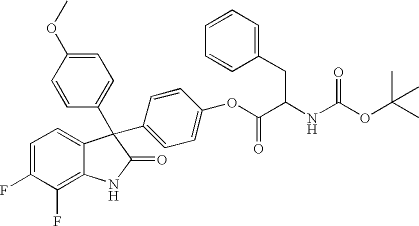

General procedure 2. Starting materials: 6,7-difluoro-3-(4-hydroxy-phenyl)-3-(4-methoxy-phenyl)-1,3-dihydro-indol-2-one and N-BOC-L-phenylalanine.¹H-NMR (CDCl₃) δ 8.60 (bs, 1H), 7.40-7.15 (m, 9H), 7.0-6.80 (m 6H), 5.09 (d, 1H), 4.82 (q, 1H), 3.80 (s, 3H), 3.23 (d, 2H), 1.46 (s, 9H).

### Preparation 3: (2S)-4-(6,7-difluoro-2-oxo-3-p-tolylindolin-3-yl )phenyl 2-(tert-butoxycarbonylamino)3-phenylpropanoate (Compound 3)

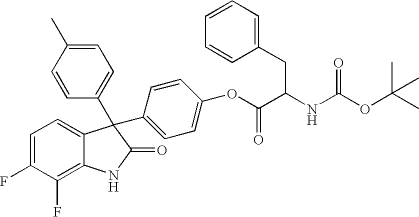

General procedure 2. Starting materials: 6,7-difluoro-3-(4-hydroxy-phenyl)-3-p-tolyl-1,3-dihydro-indol-2-one and N-BOC-L-phenylalanine. ¹H-NMR (CDCl₃) δ 7.78 (bs, 1H), 7.30-7.35 (m, 15H) 4.97 (d, 1H), 4.71 (q, 1H), 3.14 (d, 2H), 2.25( s, 3H), 1.36 (s, 9H).

### Preparation 4: (2S, 2′S)4,4′-(6,7-difluoro-2-oxoindoline-3,3-diyl )bis(4,1-phenylene)bis(2-(tert-butoxycarbonylamino)3-propanoate (Compound 4)

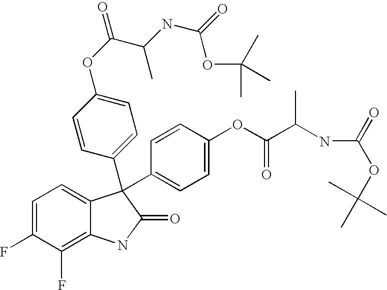

General procedure 1. Starting materials: 6,7-difluoro-3,3-bis(4-hydroxyphenyl)-indol-2-one and N-BOC-L-alanine. ¹H-NMR (CDCl3) δ 9.17 (bs, 1H), 7.17 (d, 4H) 6.97 (d, 4H), 6.85-6.70 (m, 2H), 5.07 (d, 2H), 4.46 (q, 2H), 1.45 (s, 6H), 1.38 (s, 18H).

### Preparation 5: (2S,2′S)4,4′-(6.7-difluoro-2-oxoindoline-3,3-diyl )bis(4,1-phenylene)bis(2-(tert-butoxycarbonylamino)acetate (Compound 5)

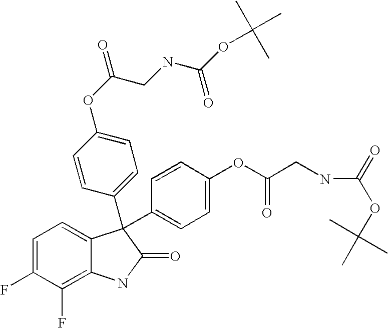

General procedure 1. Starting materials: 6,7-difluoro-3,3-bis(4-hydroxyphenyl)-indol-2-one and N-BOC-glycine. ¹H-NMR (CDCl3) δ 8.27 (bs, 1H), 7.17 (d, 4H) 6.97 (d, 4H), 6.88-6.73 (m, 2H), 5.01 (bs, 2H), 4.10 (d, 4H), 1.39 (s, 18H).

### Preparation 6: (2S,2′S)4,4′-(6,7-difluoro-2-oxoindoline-3,3-diyl)bis(4,1-phenylene)bis(2-(tert-butoxycarbonyl(methyl)amino)3-methylbutanoate (Compound 6)

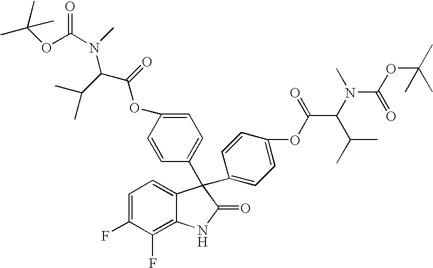

General procedure 1. Starting materials: 6,7-difluoro-3,3-bis(4-hydroxyphenyl)-indol-2-one and N-BOC-N-methyl-L-valine. ¹H-NMR (CDCl3) δ 8.15 (bs, 1H), 7.18 (d, 4H) 6.96 (d, 4H), 6.87-6.69 (m, 2H), 4.37 (dd, 2H), 2.84 (d, 3H), 2.21 (m, 2H), 1.41 (s, 18H), 1.02 (d, 6H), 0.89 (d, 6H).

### Example 1

### (2S)-4-(6,7-difluoro-3-(4-methoxyphenyl)-2-oxoindolin-3-yl)phenyl 2-aminopropanoate hydrochloride (Compound 1001)

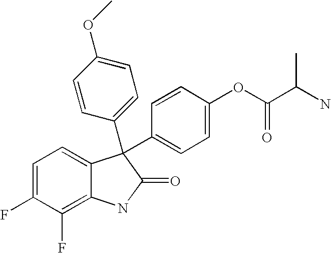

General procedure 3. Starting materials: compound 1. ¹H-NMR (DMSO-d₆) δ 11.59 (s, 1H), 8.59 (bs, 3H), 7.4-6.8 (m, 10H), 4.40 (bs. 1H) 3.73 (s, 3H), 1.56 (bs, 3H).

### Example 2

### (2S,2′S)-4,4′-(6,7-difluoro-2-oxoindoline-3.3-diyl)bis(4,1-phenylene)bis(2-aminopropanoate dihydrochloride (Compound 1002)

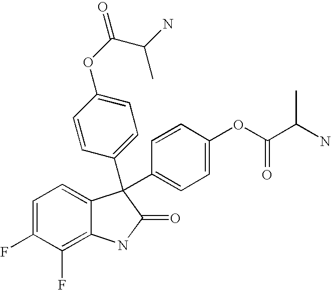

General procedure 3. Starting materials: compound 4. ¹H-NMR (DMSO-d₆) δ 11.70 (s, 1H), 8.69 (bs, 6H), 7.4-6.8 (m, 10H), 4.39 (bs. 2H), 1.55 (bs, 6H).

### Example 3

### 4,4′-(6,7-difluoro-2-oxoindoline-3,3-diyl)bis(4,1-phenylene)bis(2-aminoacetate)ditrifluoroacetic acid salt (Compound 1003)

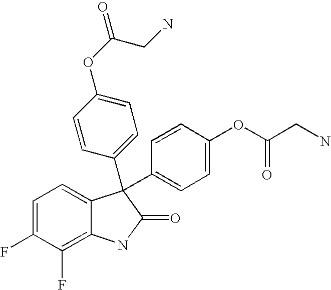

General procedure 4. Starting materials: compound 5. ¹H-NMR (DMSO-d₆) δ 11.70 (s, 1H), 8.43 (bs, 6H), 7.34-7.04 (m, 10H), 4.13 (S, 4H).

### Example 4

### (2S,2′S)-4,4′-(6,7-difluoro-2-oxoindoline-3,3-diyl)bis(4,1-phenylene)bis(3-methyl-2-(methylamino)butanoate)dihydrochloride (Compound 1004)

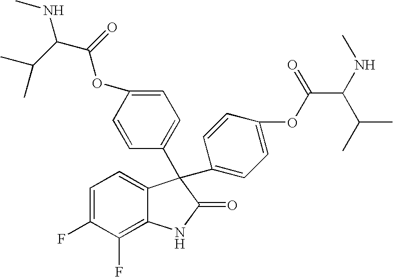

General procedure 3. Starting materials: compound 6. ¹H-NMR (DMSO-d₆) δ 11.71 (s, 1H), 9.55 (bs, 4H), 7.4-7.0 (m, 10H), 4.26 (bs. 2H), 2.66 (s, 6H), 2.48 (m, 2H), 1.17 (d, 6H); 1.05 (d, 6H).

### Example 5

### 4,4′-(6,7-difluoro-2-oxoindoline-3,3-diyl)bis(4, 1-phenylene)bis(2-dimethylamino)acetate) (Compound 1005)

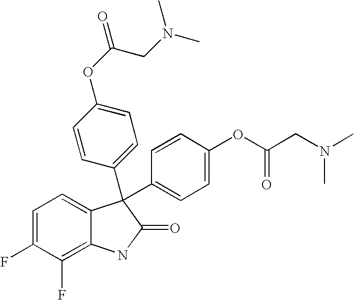

General procedure 5. Starting materials: 6,7-difluoro-3,3-bis(4-hydroxyphenyl)-indol-2-one and N.N-dimethylglycine. ¹H-NMR (DMSO-d₆) δ 11.66 (s, 1H), 7.3-7.0 (m, 10H), 3.46 (s, 2H), 2.32 (s, 6H).

### Example 6

### (2S)-4-(6,7-difluoro-3-(4-methoxyphenyl)-2-oxoindolin-3-yl)phenyl 2-aminophenylpropanoate hydrochloride (Compound 1006)

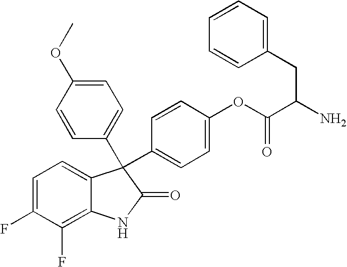

General procedure 3. Starting materials: compound 2.¹H-NMR (DMSO-d₆) δ 11.57 (s, 1H), 8.71, 7.28-7.42 (m, 5H), 7.17 (d, 2H), 7.0-7.15 (m, 4H), 6.86-6.98 (m, 4H), 4.58 (t, 1H), 3.73 (s, 3H), 3.17 (d, 2H)

General Procedure for the Following Examples

¹H NMR spectra were recorded at ambient temperature with Mercury 200 (Varian) spectrometer. The HPLC measurements were performed on an Alliance 2695 Separations Module equipped with a Waters 2487 UV detector by LiChrospher RP Select B 4.0×250 mm column. Elemental analyses were obtained with a Carlo Erba EA 11 08 instrument. Melting points were measured on a “Boetius” micro melting point apparatus and are uncorrected. Chromatographical purifications of compounds were performed by column chromatography (CC) on silicagel, 0.035-0.070 mm, (Acros) or by flash chromatography (FC) on a Biotage SP1 system with a Flash 12+M silicagel column. All the solvents were purified before use by routine techniques. To isolate reaction products, the solvents were removed by evaporation using a vacuum rotary evaporator, the water bath temperature not exceeding 40° C.

Various reagents were purchased from Acros Organics (Janssens Pharmaceuticalaan 3A, 2440 Geel, Belgium).

### Example 7

### 6,7-Difluoro-3-(4-fluorophenyl)-1-(2-hydroxyethyl)-3-(4-hydroxyphenyl)-1,3-dihydro-2H-indol-2-one (Compound 1007)

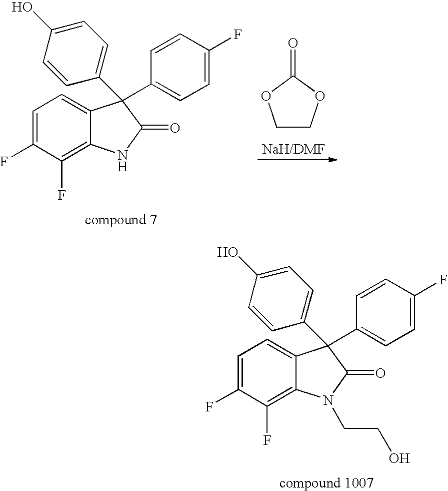

To a solution of 6,7-difluoro-3-(4-fluorophenyl)-3-(4-hydroxyphenyl)-1,3-dihydro-2H-indol-2-one (compound 7) (0.200g, 0.56 mmol) in dimethylformamide (0.5 mL) successively 60% NaH in mineral oil (0.010 g, 0.25 mmol) and 1,3-dioxalan-2-one (0.054 g, 0.62 mmol) were added. The reaction mixture was stirred at 140° C. for 18 h, cooled, and concentrated in vacuo. The residue was purified by CC (15 g of SiO₂) with dichloromethane-ethanol (100:5) as eluent and twice by FC with dichlormethane-ethanol (gradient from 100:0 to 80:20) as eluent to give the title compound 1007 (0.070 g, 31%) as a foam, m.p. 80-83° C. 1H-NMR (DMSO-d₆, HMDSO) δ: 3.65 (m, 2H); 3.93 (m, 2H); 4.94 (t, J=5.4 Hz, 1H); 6.70 (d, J=8.8 Hz, 2H); 6.95 (d, J=8.8 Hz, 2H); 7.03-7.22 (m, 6H); 9.50 (s, 1H). Anal. Calcd for C₂₂H₁₆F₃NO₃.0.5H₂O, %: C, 64.71; H, 4.20; N, 3.43. Found, %: C, 64.76; H, 4.29; N, 3.58.

### Example 8

### 4-[6,7-Difluoro-3-(4-fluorophenyl)-2-oxo-2,3-dihydro-1H-indol-3-yl]phenyl acrylate (Compound 1008), 4-[6,7-Difluoro-3-(4-fluorophenyl)-2-oxo-2,3-dihydro-1H-indol-3-yl]phenyl 3-morpholinopropanoate (Compound 1009) and 4-[6,7-Difluoro-3-(4-fluorophenyl)-2-oxo-2,3-dihydro-1H-indol-3-yl]phenyl 3-(4-methylpiperazino)propanoate (Compound 1010)

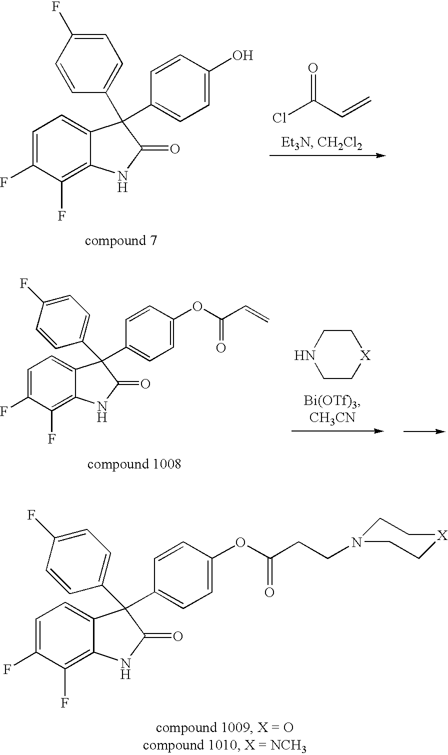

To a solution of 6,7-difluoro-3-(4-fluorophenyl)-3-(4-hydroxyphenyl)-1,3-dihydro-2H-indol-2-one (compound 7) (0.180g, 0.5 mmol) and triethylamine (0.22 mL, 1.58 mmol) in dichloromethane (5 mL) at −10° C. acryloyl chloride (0.042 mL, 0.52 mmol) was added and the resulting mixture was stirred at this temperature for 3 h. The reaction mixture was supplemented with dichloromethane (45 mL), washed with sat. NaCl solution (4×10 mL), and dried (Na₂SO₄). The solvent was evaporated and the residue was purified by FC with a mixture of petroleum ether and ethyl acetate-dichloromethane, 1:1 (gradient from 100:0 to 30:70) as eluent to give the title compound 1008 (0.058 g, 27%) as an oil which solidified on standing. ¹H-NMR (CDCl₃, HMDSO) δ: 6.01 (dd, J=1.4, 10.2 Hz, 1H); 6.30 (dd, J=10.2, 17.2 Hz, 1H); 6.59 (dd, J=1.4, 17.2 Hz, 1H); 6.78-6.93 (m, 2H); 6.99 (t, J=8.8 Hz, 2H); 7.09 (d, J=8.6 Hz, 2H); 7.21 (dd, J=5.3, 8.8 Hz, 2H); 7.24 (d, J=8.6 Hz, 2H); 8.62 (s, 1H).

To a solution of 4-[6,7-difluoro-3-(4-fluorophenyl)-2-oxo-2,3-dihydro-1H-indol-3-yl]phenyl acrylate (compound 1008) (0.052 g, 0.13 mmol) in dry acetonitrile (3 mL) morpholine (0.012 mL, 0.14 mmol) followed by bismuth(III) trifluoromethanesulfonate (0.0016 g, 0.0024 mmol) were added and the reaction mixture was stirred overnight at room temperature. The precipitated solid was filtered, crystallized from acetonitrile, and dried to give the title compound 1009 (0.032 g, 50%) as white crystals, m.p. 174-175° C. 1H-NMR (DMSO-d₆, HMDSO) δ: 2.33-2.46 (m, 4H, overlapped with DMSO); 2.57-2.87 (m, 4H, overlapped with DMSO); 3.50-3.62 (m, 4H); 6.97-7.26 (m, 10H); ˜11-12 (b s, 1H). Anal. Calcd for C₂₇H₂₃F₃N₂O₄.0.5 H₂O.0, %: C, 64.15; H, 4.79; N, 5.54. Found, %: C, 64.04; H, 4.43; N, 5.69.

Compound 1010 was prepared from 4-[6,7-difluoro-3-(4-fluorophenyl)-2-oxo-2,3-dihydro-1H-indol-3-yl]phenyl acrylate (compound 1008) and 1-methylpiperazine in the same manner as for compound 1009 above in 61% yield was prepared. M.p. 149-151° C. 1H-NMR (DMSO-d₆, HMDSO) δ: 2.13 (s, 3H); 2.30 (m, 4H); 2.40 (m, 4H); 2.68 (m, 4H); 6.97-7.27 (m, 10H); 11.61 (b s, 1H). Anal. Calcd for C₂₈H₂₆F₃N₃O₃, %: C, 66.00; H, 5.14; N, 8.25. Found, %: C, 65.82; H, 4.96; N, 8.42.

### Example 9

### [3-(4-{[(Isopropoxycarbonyl)oxy]methoxy}phenyl)-3-(4-methoxyphenyl)-2-oxo-2,3-dihydro-1H-indol-1-yl]methyl isopropyl carbonate (Compound 1011)

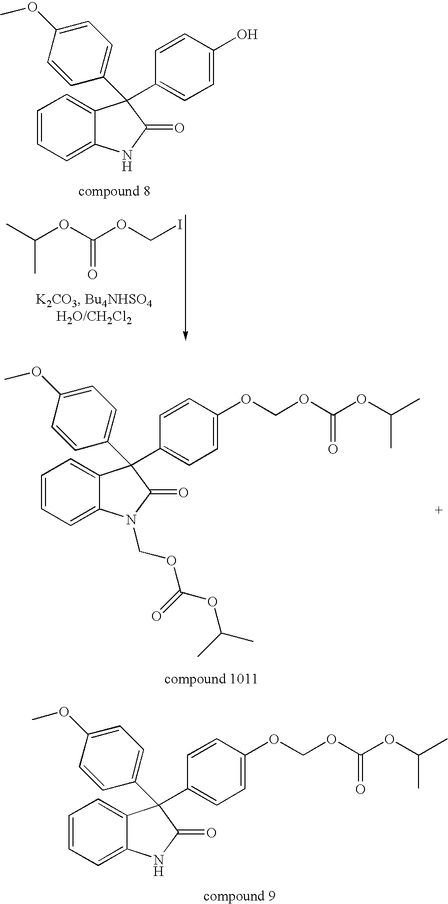

To a suspension of 3-(4-hydroxyphenyl)-3-(4-methoxyphenyl)-1,3-dihydro-2H-indol-2-one (compound 8) (0.16 g, 0.48 mmol) in water (2.5 mL) K₂CO₃ (0.21 g, 1.5 mmol) was added and the mixture was stirred at r.t. for 5 min. Then Bu₄NHSO₄ (0.17 g, 0.5 mmol), dichloromethane (2.5 mL), and iodomethyl isopropyl carbonate (0.16 g, 0.65 mmol) were added and the resulting mixture was vigorously stirred at r.t. for 24 h. (The preparation of iodomethyl isopropyl carbonate was described in literature (Thomas, J. D.; Sloan, K. B. Tetrah. Lett. 2007, 48 (1), 109-112).) The aqueous layer was separated, the organic layer was supplemented with dichloromethane (10 mL), washed with sat. NaCl solution, and dried (Na₂SO₄). The solvent was evaporated and the residue was purified by CC (20 g of SiO₂) with dichloromethane-ethanol (100:1) as eluent to give the title compound compound 1011 (0.030 g, 11%) and compound 10 (0.11 g, 51%). Compound 1011: m.p. 47-49° C. 1H-NMR (DMSO-d₆, HMDSO) δ: 1.20 (d, J=6.2 Hz, 6H); 1.21 (d, J=6.2 Hz, 6H); 3.72 (s, 3H); 4.79 (septet, J=6.2 Hz, 1H); 4.79 (septet, J=6.2 Hz, 1H); 5.76 (s, 2H); 5.83 (s, 2H); 6.89 (d, J=8.8 Hz, 2H); 7.02 (d, J=9.0 Hz, 2H); 7.05 (d, J=8.8 Hz, 2H); 7.09 (d, J=9.0 Hz, 2H); 7.11-7.22 (m, 1H); 7.25-7.45 (m, 3H). Anal. Calcd for C₃₁H₃₃NO₉, %: C, 66.06; H, 5.90; N, 2.49. Found, %: C, 66.16; H, 5.87; N, 2.51. Compound 9: m.p. 63-65° C. 1H-NMR (DMSO-d₆, HMDSO) δ: 1.21 (d, J=6.2 Hz, 6H); 3.71 (s, 3H); 4.79 (septet, J=6.2 Hz, 1H); 5.75 (s, 2H); 6.88 (d, J=8.9 Hz, 2H); 6.91-7.15 (m, 8H); 7.16-7.29 (m, 2H); 10.71 (s, 1H). Anal. Calcd for C₂₆H₂₅NO₆, %: C, 69.79; H, 5.63; N, 3.13. Found, %: C, 69.68; H, 5.60; N, 3.13.

### Example 10

### Benzyl 2-[3-{4-[2-(benzyloxy)-2-oxoethoxy]phenyl}-3-(4-methoxyphenyl)-2-oxo-2,3-dihydro-1H-indol-1-yl]acetate (Compound 1012)

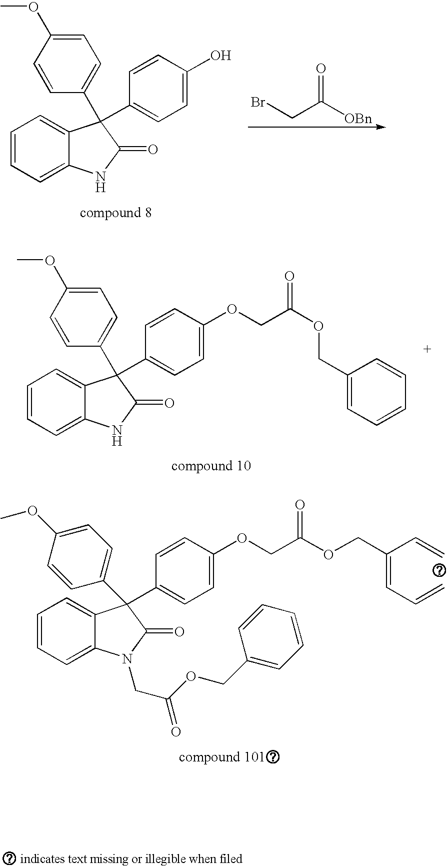

Compound 10 and compound 1012 were prepared from 3-(4-hydroxyphenyl)-3-(4-methoxyphenyl)-1,3-dihydro-2H-indol-2-one (compound 8) and benzyl 2-bromoacetate by the same protocol as for compound 1011 above in 15% and 78% yields, accordingly. Compound 10: m.p. 76-78° C. 1H-NMR (DMSO-d₆, HMDSO) δ: 3.72 (s, 3H); 4.83 (s, 2H); 5.17 (s, 2H); 6.87 (d, J=8.9 Hz, 2H); 6.87 (d, J=8.9 Hz, 2H); 6.90-7.02 (m, 2H); 7.04 (d, J=8.9 Hz, 2H); 7.05 (d, J=8.9 Hz, 2H); 7.16-7.28 (m, 2H); 7.33 (s, 5H); 10.69 (s, 1H). Anal. Calcd for C₃₀H₂₅NO₅, %: C, 75.14; H, 5.25; N, 2.92. Found, %: C, 74.84; H, 5.32; N, 2.83. Compound 1012: m.p. 57-58° C. 1H-NMR (CDCl₃, HMDSO) δ: 3.75 (s, 3H); 4.56 (s, 2H); 4.60 (s, 2H); 5.17 (s, 2H); 5.21 (s, 2H); 6.69-6.80 (m, 5H); 7.03-7.39 (m, 17H). Anal. Calcd for C₃₉H₃₃NO₇, %: C, 74.63; H, 5.30; N, 2.23. Found, %: C, 74.48; H, 5.30; N, 2.19.

### Example 11

### 4-(3-{4-[(Anilinocarbonyl)oxy]phenyl}-6,7-difluoro-2-oxo-2,3-dihydro-1H-indol-3-yl)phenyl N-phenylcarbamate (Compound 1013)

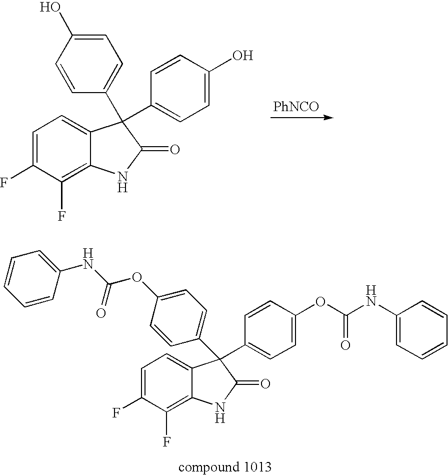

To a suspension of 6,7-difluoro-3,3-bis(4-hydroxyphenyl)-1,3-dihydro-2H-indol-2-one (0.35 g, 1.0 mmol) in dry toluene under argon atmosphere phenyl isocyanate (0.24 mL, 2.2 mmol) was added and the mixture was stirred at reflux for 3 h. The precipitated solid was filtered, purified by CC (30 g of SiO₂) with dichloromethane-ethanol (100:1) as eluent and crystallized from acetonitrile to give the title compound 1013 (0.40 g, 68%), m.p. 163-165° C. 1H-NMR (DMSO-d₆, HMDSO) δ: 6.94-7.38 (m, 2H); 7.04 (t, J=7.2 Hz, 2H); 7.23 (s, 8H); 7.32 (t, J=7.8 Hz, 4H); 7.49 (d, J=8.0 Hz, 4H); 10.25 (br s, 2H); 11.67 (br s, 1H). Anal. Calcd for C₃₄H₂₃F₂N₃O₅.0.7 H₂O, %: C, 67.59; H, 4.07; N, 6.95. Found, %: C, 67.61; H, 3.87; N, 7.30.

### Example 12

### 4-[6,7-Difluoro-3-(4-fluorophenyl)-2-oxo-2.3-dihydro-1H-indol-3-yl]phenyl N,N-dimethylcarbamate (Compound 1014)

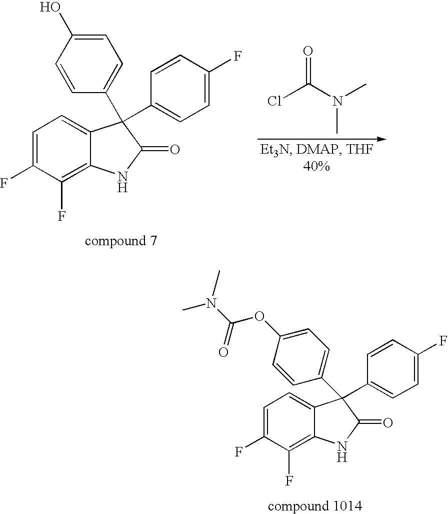

To a solution of 6,7-difluoro-3-(4-fluorophenyl)-3-(4-hydroxyphenyl)-1,3-dihydro-2H-indol-2-one (compound 7) (0.110 g, 0.31 mmol), TEA (0.13 mL, 0.93 mmol), and DMAP (0.0076 g, 0.062 mmol) in THF (7 mL) was added dimethylcarbamyl chloride (0.033 mL, 0.34 mmol) and the reaction mixture was refluxed for 3 h. The solvent was evaporated, the residue was dissolved in CH₂Cl₂ (40 mL), washed with sat. NaCl solution (2×10 mL), and dried (Na₂SO₄). The solvent was evaporated and the residue was purified by FC with CHCl₃—MeOH (gradient from 100:0 to 80:20) as eluent to give the title compound 1014 (0.053 g, 40%) as a white solid, m.p. 98-100° C. ¹H-NMR (DMSO-d₆, HMDSO) δ: 3.00 (s, 3H); 3.08 (s, 3H); 6.77-7.12 (m, 6H); 7.14-7.25 (m, 4H); 8.22 (s, 1H). Anal. Calcd for C₂₃H₁₇F₃N₂O₃ as if it contained 5.⁵% of ballast impurities, %: C, 61.22; H, 3.80; N, 6.21. Found, %: C, 61.25; H, 3.80; N, 6.07.

### Example 13

### 4-[6-Fluoro-3-(4-fluorophenyl)-7-methyl-2-oxo-2.3-dihydro-1H-indol-3-yl]phenyl N,N-dimethylcarbamate (Compound 1015) and 4-[6-Fluoro-3-(4-fluorophenyl)-7-methyl-2-oxo-2,3-dihydro-1H-indol-3-yl]phenyl acrylate (Compound 1016)

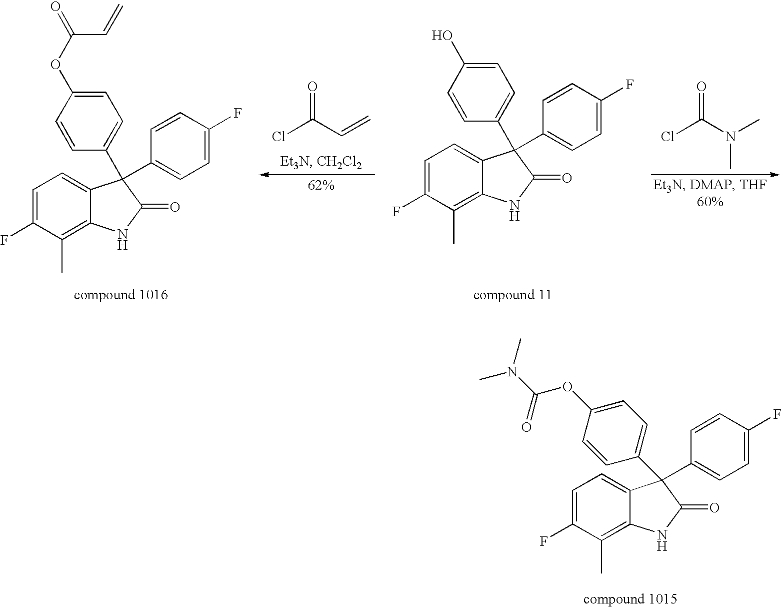

Compound 1015 was prepared from 6-fluoro-3-(4-fluorophenyl)-3-(4-hydroxyphenyl)-7-methyl-1,3-dihydro-2H-indol-2-one (compound 11) in the same manner as for compound 1014 above in 60% yield. M.p. 263-265° C. ¹H-NMR (DMSO-d₆, HMDSO) δ: 2.19 (d, J=1.4 Hz, 3H); 2.89 (s, 3H); 3.01 (s, 1H); 6.82 (dd, J=8.4, 10.2 Hz, 1H); 7.03-7.23 (m, 9H); 11.08 (s, 1H).

To a solution of 6-fluoro-3-(4-fluorophenyl)-3-(4-hydroxyphenyl)-7-methyl-1,3-dihydro-2H-indol-2-one (compound 11) (0.200g, 0.6 mmol) and triethylamine (0.24 mL, 1.7 mmol) in dichloromethane (5 mL) at ice bath temperature acryloyl chloride (0.046 mL, 0.60 mmol) was added and the resulting mixture was stirred at r.t. overnight. The reaction mixture was supplemented with dichloromethane (55 mL), washed with sat. NaCl solution (2 ×10 mL), and dried (Na₂SO₄). The solvent was evaporated and the residue was purified by FC with a mixture of petroleum ether and ethyl acetate-dichloromethane, 1:1 (gradient from 100:0 to 30:70) as eluent to give the title compound 1016 (0.15 g, 62%) as a white solid, m.p. 223-225° C. ¹H-NMR (CDCl₃, HMDSO) δ: 2.20 (d, J=1.3 Hz, 3H); 6.15 (dd, J=2.2, 9.5 Hz, 1H); 6.39 (dd, J=9.5, 17.2 Hz, 1H); 6.52 (dd, J=2.2, 17.2 Hz, 1H); 6.83 (dd, J=8.3, 10.2 Hz, 1H); 7.08-7.28 (m, 9H); 11.11 (s, 1H).

1. A compound of the general formula (I) 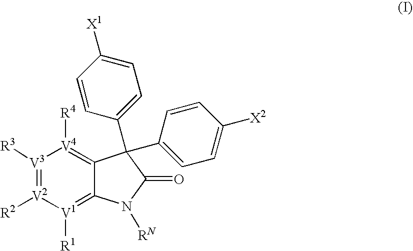
wherein
each of X¹ and X² independently represents a prodrug group of any of the types (i)-(vi)
(i) —O—C(═O)—Z, wherein Z is selected from substituted C₁₋₆-alkyl and —CH(R⁶)N(R⁷)R⁸;
(ii) —O—C(═O)—O—Y, wherein Y is selected from optionally substituted C₁₋₆-alkyl, or —O—Y represents 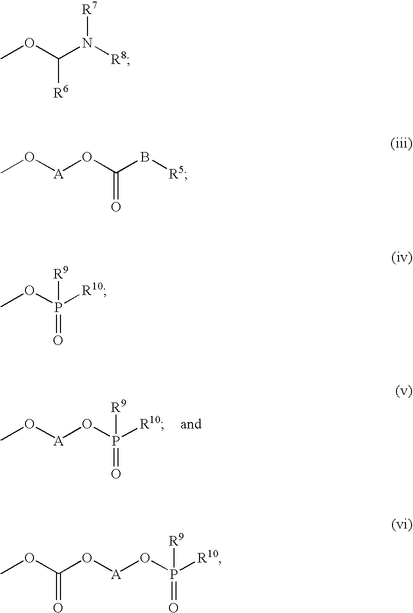
wherein A is selected from optionally substituted C₁₋₆-alkylidene and optionally substituted benzylidene,
B is selected from a single bond, —O— and —S—,
R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug groups (iii) and (vii) may represent an optionally N-substituted amino acid;
R⁶ is selected from hydrogen, optionally substituted C₁₋₆-alkyl, optionally substituted C₂₋₆-alkenyl, aryl, heterocyclyl, and heteroaryl, wherein any aryl, heterocyclyl and heteroaryl may be optionally substituted, or R⁶ and R⁸ together with the intervening carbon and nitrogen atoms to which they are attached form a heterocyclic ring;
R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted, or R⁷ and R⁸ together with the nitrogen atoms to which they are attached form a heterocyclic ring; and
R⁹ is selected from hydrogen, hydroxy, optionally substituted C₁₋₆-alkyl, optionally substituted C₁₋₆-alkoxy, and optionally substituted C₂₋₆-alkenyloxy;
R¹⁰ is selected from hydroxy, optionally substituted C₁₋₆-alkyl, optionally substituted C₁₋₆-alkoxy, optionally substituted C₂₋₆-alkenyloxy; aryloxy, heterocyclyloxy, and heteroaryloxy, wherein any aryl, heterocyclyl and heteroaryl may be optionally substituted;
provided that R⁹ and R¹⁰ are not both selected from hydroxy and C₁₋₆-alkoxy;
or is selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted;
R^{N} represents a prodrug group of any of the types (vii)-(viii) 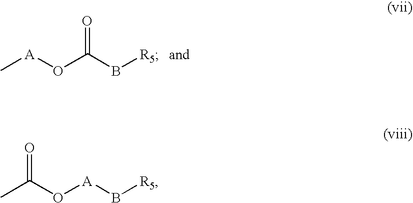
wherein A, B and R⁵ are as defined above for prodrug group (iii);
or is selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);
with the proviso that the compound comprises at least one of the prodrug groups (i)-(viii);
V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;
R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R¹ and R² together with the carbon atoms to which they are attached form a ring;
with the proviso that when each of V¹, V², V³ and V⁴ represents a carbon atom, then R^{N}, R¹, R², R³, and R⁴ are not all hydrogen; and
pharmaceutically acceptable salts thereof.

2. The compound according to claim 1, wherein at least one of X¹ and X² represents a prodrug group (i) —O—C(═O)—Z.

3-5. (canceled)

6. The compound according to claim 1, wherein at least one of X¹ and X² represents a prodrug group (ii) —O—C(═O)—O—Y.

7. (canceled)

8. The compound according to claim 1, wherein at least one of X¹ and X² represents a prodrug group (iii)

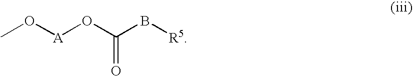

9-11. (canceled)

12. The compound according to claim 1, wherein at least one of X¹ and X² represents a prodrug group of any of the types (iv)-(vi)

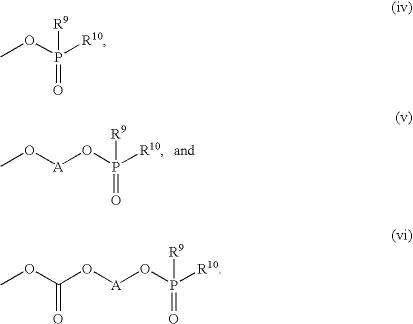

13-14. (canceled)

15. The compound according to claim 1, wherein R^{N} represents a prodrug group or any of the types (vii)-(viii)

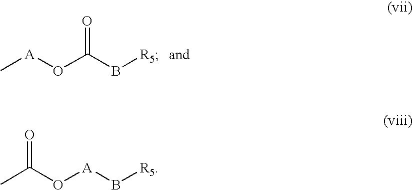

16-17. (canceled)

18. The compound according to claim 1, wherein each of V¹, V², V³, and V⁴ represents a carbon atom.

19. The compound according to claim 1, wherein R¹, R², R³ and R⁴ are not all hydrogen.

20-22. (canceled)

23. The compound according to claim 1, wherein R^{N} is hydrogen.

24. The compound according to claim 1, wherein
each of V¹, V², V³, and V⁴ represents a carbon atom;
R¹ and R² are both fluoro;
R³ and R⁴ are all hydrogen;
R^{N} is hydrogen;
at least one of X¹ and X² represents a prodrug group (i) —O—C(═O)—CH(R⁶)—N(R⁷)R⁸,
wherein R⁶ is selected from hydrogen(glycine), methyl(alanine), 2-propyl(valine), 2-methyl-1-propyl(leucine), 2-butyl(isoleucine), methylthioethyl(methionine), benzyl(phenylalanine), 3-indolylmethyl(tryptophan), hydroxymethyl(serine), 1-hydroxyethyl(threonine), mercaptomethyl(cysteine), 4-hydroxybenzyl(tyrosine), aminocarbonylmethyl(asparagine), 2-aminocarbonylethyl(glutamine), carboxymethyl(aspartic acid), 2-carboxyethyl(glutamic acid), 4-amino-1-butyl(lysine), 3-guanidino-1-propyl(arginine), and 4-imidazolylmethyl(histidine), or R⁶ and R⁸ together with the intervening carbon and nitrogen atoms to which they are attached form a pyrrolidine ring (proline);
R⁷ is hydrogen and R⁸ is selected from hydrogen, optionally substituted C₁₋₆-alkyl, optionally substituted C₁₋₆-alkoxycarbonyl, optionally substituted C₁₋₆-alkylcarbonyl, mono- and di(C₁₋₆-alkyl)aminocarbonyl, C₁₋₆-alkylsulphonyl, C₁₋₆-alkylsulphinyl, aryl, arylcarbonyl, heterocyclyl, heterocyclyloxy, heteroaryl, and heteroaryloxy; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or R⁷ and R⁸ together with the nitrogen atoms to which they are attached form a heterocyclic ring; and
any other of X¹ or X² is selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted.

25. A compound of the general formula (Ia) 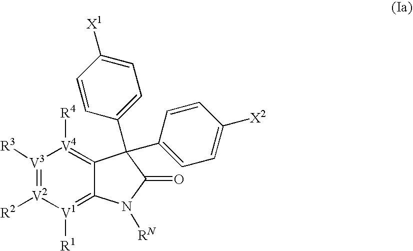
wherein
at least one of X¹, X² and R^{N} represent a prodrug group comprising an amino acid moiety,
any of X¹ and X² not being a prodrug group independently being selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)-aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted;
and R^{N} not being a prodrug group being selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);
V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;
R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R¹ and R² together with the carbon atoms to which they are attached form a ring;
with the proviso that when each of V¹, V², V³ and V⁴ represents a carbon atom, then R^{N}, R¹, R², R³, and R⁴ are not all hydrogen; and
pharmaceutically acceptable salts thereof.

26-27. (canceled)

28. A compound selected from the group consisting of
(2S)-4-(6,7-difluoro-3-(4-methoxyphenyl)-2-oxoindolin-3-yl)phenyl 2-aminopropanoate hydrochloride,
(2S,2′S)-4,4′-(6,7-difluoro-2-oxoindoline-3,3-diyl)bis(4,1-phenylene)bis(2-aminopropanoate dihydrochloride,
4,4′-(6,7-difluoro-2-oxoindoline-3,3-diyl)bis(4,1-phenylene)bis(2-aminoacetate)ditrifluoroacetic acid salt,
(2S,2′S)-4,4′-(6,7-difluoro-2-oxoindoline-3,3-diyl)bis(4,1-phenylene)bis(3-methyl-2-(methylamino)butanoate)dihydrochloride,
4,4′-(6,7-difluoro-2-oxoindoline-3,3-diyl)bis(4,1-phenylene)bis(2-dimethylamino)acetate),
(2S)-4-(6,7-difluoro-3-(4-methoxyphenyl)-2-oxoindolin-3-yl)phenyl 2-aminophenylpropanoate hydrochloride,
4-(3-(4-chlorophenyl)-6,7-difuoro-2-oxoindolin-3-yl)phenyl(2-morpholinoethoxy)methyl carbonate,
(7-fluoro-3,3-bis(4-hydroxyphenyl)-6-methyl-2-oxoindolin-1-yl)methyl 2-morpholinoethyl carbonate,
(4,4′-(6-fluoro-7-methyl-2-oxoindoline-3,3-diyl)bis(4,1-phenylene)bis(oxy)bis(methylene)bis(2-morpholinoethyl) dicarbonate, and
4-(3-(4-chlorophenyl)-6,7-dimethyl-2-oxoindolin-3-yl)phenyl methyl methylphosphonate.

29. A compound of the general formula (Ib) 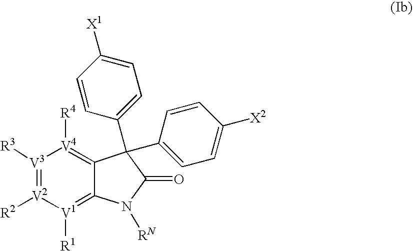
wherein
each of X¹ and X² independently represents a prodrug group of any of the types (ix)-(x)
(ix) —O—C(═O)—Z, wherein Z is selected from optionally substituted C₁₋₆-alkenyl and —N(R⁷)R⁸; and
(x) —O—CH₂—C(═O)—Y, wherein Y is selected from optionally substituted C₁₋₆-alkyl;
wherein R⁷ and R⁸ are independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted, or R⁷ and R⁸ together with the nitrogen atoms to which they are attached form an optionally substituted heterocyclic ring;
or is selected from hydrogen, hydroxy, optionally substituted C₁₋₆ alkoxy, optionally substituted C₁₋₆ alkyl, optionally substituted C₂₋₆ alkenyl, carboxy, optionally substituted C₁₋₆-alkoxycarbonyl, C₁₋₆-alkylcarbonyloxy, optionally substituted C₁₋₆ alkylcarbonyl, formyl, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, carbamoyl, mono-and di(C₁₋₆-alkyl)aminocarbonyl, mercapto, optionally substituted C₁₋₆-alkylthio, C₁₋₆-alkylsulfonyl, mono- and di(C₁₋₆-alkyl)aminosulfonyl, cyano, halogen, aryl, aryloxy, arylamino, arylcarbonyl, heterocyclyl, heterocyclyloxy, heterocyclylamino, heterocycylcarbonyl, heteroaryloxy, heteroarylamino, heteroarylcarbonyl, where any C₁₋₆-alkyl as an amino substituent is optionally substituted with hydroxyl, C₁₋₆-alkoxy, amino, mono and di(C₁₋₆-alkyl)amino, carboxy, C₁₋₆-alkylcarbonylamino, C₁₋₆ alkylaminocarbonyl or halogen(s) and wherein any aryl, heterocyclyl and heteroaryl may be optionally substituted;
R^{N} represents a prodrug group of any of the types (xi)-(xii)
(xi) -A-C(═O)—B—R⁵ ; and
(xii) —(CH₂—CH₂—O)₁₋₁₀—R⁵,
wherein A is selected from optionally substituted C₁₋₆-alkylidene and optionally substituted benzylidene,
B is selected from a single bond, —O— and —S—, and
R⁵ is selected from hydrogen, optionally substituted C₁₋₆-alkyl, optionally substituted C₁₋₆-alkoxy, optionally substituted C₁₋₆-alkoxycarbonyl, optionally substituted C₁₋₆-alkylcarbonyl, mono- and di(C₁₋₆-alkyl)aminocarbonyl, amino, C₁₋₆-alkylcarbonylamino, mono- and di(C₁₋₆-alkyl)amino, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; or —C(═O)—B—R⁵ in prodrug group (xi) may represent an optionally N-substituted amino acid;
or is selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s);
with the proviso that the compound comprises at least one of the prodrug groups (ix)-(xii);
V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;
R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R¹ and R² together with the carbon atoms to which they are attached form a ring;
with the proviso that when each of V¹, V², V³ and V⁴ represents a carbon atom, then R^{N}, R¹, R², R³, and R⁴ are not all hydrogen; and
pharmaceutically acceptable salts thereof.

30-33. (canceled)

34. A compound selected from the group consisting of
6,7-Difluoro-3-(4-fluorophenyl)-1-(2-hydroxyethyl)-3-(4-hydroxyphenyl)-1,3-dihydro-2H-indol-2-one,
4-[6,7-Difluoro-3-(4-fluorophenyl)-2-oxo-2,3-dihydro-1H-indol-3-yl]phenyl 3-morpholinopropanoate,
4-[6,7-Difluoro-3-(4-fluorophenyl)-2-oxo-2,3-dihydro-1H-indol-3-yl]phenyl 3-(4-methylpiperazino)propanoate,
[3-(4-{[(Isopropoxycarbonyl)oxy]methoxy}phenyl)-3-(4-methoxyphenyl)-2-oxo-2,3-dihydro-1H-indol-1-yl]methyl isopropyl carbonate,
Benzyl 2-[3-{4-[2-(benzyloxy)-2-oxoethoxy]phenyl}-3-(4-methoxyphenyl)-2-oxo-2,3-dihydro-1H-indol-1-yl]acetate,
4-(3-{4-[(Anilinocarbonyl)oxy]phenyl}-6,7-difluoro-2-oxo-2,3-dihydro-1H-indol-3-yl)phenyl N-phenylcarbamate,
4-[6,7-Difluoro-3-(4-fluorophenyl)-2-oxo-2,3-dihydro-1H-indol-3-yl]phenyl N,N-dimethylcarbamate,
4-[6-Fluoro-3-(4-fluorophenyl)-7-methyl-2-oxo-2,3-dihydro-1H-indol-3-yl]phenyl N,N-dimethylcarbamate, and
4-[6-Fluoro-3-(4-fluorophenyl)-7-methyl-2-oxo-2,3-dihydro-1H-indol-3-yl]phenyl acrylate.

35. A pharmaceutical composition comprising a compound of the general formula (I) as defined in claim 1 and a pharmaceutically acceptable carrier.

36-39. (canceled)

40. A method of treating a mammal suffering from or being susceptible to cancer, the method comprising administering to the mammal a therapeutically effective amount of a compound of the general formula (I) as defined in claim 1.

41. A method of treating a mammal suffering from or being susceptible to cancer, the method comprising administering to the mammal a therapeutically effective amount of a compound of the general formula (Ia) as defined in claim 25.

42. A method of treating a mammal suffering from or being susceptible to cancer, the method comprising administering to the mammal a therapeutically effective amount of a compound of the general formula (Ib) as defined in claim 29.
